SEQUENCE LISTING (11 sequences)

SEQ ID NO: 1 - RNA, 23 nt, Homo sapiens
uguaguguuuccuacuuuaugga

SEQ ID NO: 2 - RNA, 87 nt, Homo sapiens
gacagugcagucacccauaaaguagaaagcacuacuaacagcacuggaggguguaguguuuccuacuuuauggaugaguguacugug

SEQ ID NO: 3 - RNA, 24 nt, Homo sapiens
cauaaaguaggcaacaucuaccuu

SEQ ID NO: 4 - RNA, 24 nt, Homo sapiens
ucacaaccuccuagaaagaguaga

SEQ ID NO: 5 - RNA, 24 nt, Homo sapiens
uacucuuucuagaagguggugcuu

SEQ ID NO: 6 - DNA, 21 bp, Homo sapiens
tctctctaatcagccctctgg

SEQ ID NO: 7 - DNA, 19 bp, Homo sapiens
actcggggttcgagaagat

SEQ ID NO: 8 - DNA, 20 bp, Homo sapiens
acctccagggacaggatatg

SEQ ID NO: 9 - DNA, 20 bp, Homo sapiens
aacacgcaggacaggtacag

SEQ ID NO: 10 - DNA, 22 bp, Homo sapiens
gagtaccgcaaacgctttatgc

SEQ ID NO: 11 - DNA, 19 bp, Homo sapiens
atctggccgaggcttttct

(19) United States
(12) Patent Application Publication
(10) Pub. No.: US 2022/0313723 A1
(43) Pub. Date: Oct. 6, 2022

(54) EXTRACELLULAR VESICLES CONTAINING MIR-142-3P TO TREAT FIBROSING DISEASES

(71) Applicants: Université de Liège, Liège (BE); Centre Hospitalier Universitaire de Liège, Liège (BE)

(72) Inventors: Julien Guiot, Verlaine (BE); Ingrid Struman, Boncelles (BE); Makon-Sébastien Njock, Angleur (BE)

(21) Appl. No.: 17/597,533

(22) PCT Filed: Jul. 16, 2020
(86) PCT No.: PCT/EP2020/070215

§ 371 Date: Jan. 10, 2022

(30) Foreign Application Priority Data
Jul. 16, 2019 (EP) 19186566.6

(51) Int. Cl. A61K 31/713; A61P 11/00; A61K 35/15; A61K 35/33

(52) U.S. Cl. CPC A61K 31/713; A61P 11/00; A61K 35/15; A61K 35/33

(57) ABSTRACT

The present invention relates to pharmaceutical formulations comprising extracellular vesicles wherein the content of the vesicles comprises microRNA miR-142-3p, methods for producing the pharmaceutical formulations, and uses of the formulations to treat fibro-inflammatory diseases in a subject.

### FIELD OF THE INVENTION

The invention is situated in the field of pharmaceutical formulations suitable for treating fibrosing diseases. More particularly, aspects of the invention are situated in the field of pharmaceutical formulations comprising extracellular vesicles, means to produce these and uses thereof.

### BACKGROUND OF THE INVENTION

Idiopathic pulmonary fibrosis (IPF) is a progressive fibrosing interstitial lung disease of unknown etiology and cure which leads rapidly to death within 2-3 years after diagnosis (Guiot et al., (2017), PLoS One, 12:e0171344; King et al., (2011), Lancet, 378:1949-1961; Noble et al., (2011), Lancet (London, England), 377:1760-1769; Raghu et al., (2011), Am. J. Respir. Crit. Care Med., 183:788-824). It is characterized by the progressive and irreversible destruction of lung architecture caused by fibrotic “scar” formation that ultimately leads to organ malformation, disruption of gas exchange, and death from respiratory failure (Lederer and Martinez, (2018), N. Engl. J. Med., 378:1811-1823; Wynn, (2011), J. Exp. Med., 208:1339-1350). Its pathophysiology is still not well known although recent studies suggest this disease results from aberrant dysregulated wound healing response following chronic alveolar epithelial injury and aberrant proliferation of fibroblasts (Wilson and Wynn, (2009), Mucosal Immunol., 2:103-121). The pathological hallmarks include the infiltration of inflammatory cells (e.g. neutrophils, macrophages, lymphocytes) and excessive secretion of pro-fibrotic mediators (including Transforming Growth Factor β1 (TGF-β1)), aberrant activation of epithelial mesenchymal transition, proliferation and differentiation of fibroblasts into myofibroblasts, leading to excess deposition of extracellular matrix components (e.g. collagen, fibronectin) within the lung interstitium (Wynn, (2011), J. Exp. Med., 208:1339-1350).

Genetic predispositions and environmental factors leading to epigenetic modifications (such as smoking, gastro-oesophageal reflux or viral infections) as well as cell senescence associated with ageing appear to be key factors in IPF pathophysiology (Alvarez et al., (2017), Am. J. Physiol. Lung Cell. Mol. Physiol., 313:L1164—L1173; Guiot et al., (2017), Clin. Epigenetics, 9; Kropski et al., (2013), Dis. Model. Mech., 6:9-17; Mora et al., (2017), Nat. Rev. Drug Discov., 16:755-772). Management of clinical symptoms has been improved since the discovery of new efficient therapeutic agents, pirfenidone and nintedanib (Noble et al., (2011), Lancet (London, England), 377:1760-1769; Richeldi et al., (2011), N. Engl. J. Med., 365:1079-1087). Yet, these therapies only slow disease progression or acute exacerbation risk and do not provide a definitive cure. Novel therapeutics are desperately needed. In terms of key mediators involved in promoting aberrant fibrogenesis, the pleiotropic cytokine Transforming Growth Factor β1 (TGF-β1) was shown to play a central role by modulating the epithelial and fibroblast phenotype and function through its downstream signaling pathway. There has therefore been a collective effort to develop therapeutic strategies to treat fibrosis by targeting TGF-β1 signaling (Akhurst, (2017), Cold Spring Harb. Perspect. Biol., 9:a022301).

MicroRNAs (miRs) have emerged as potent modulators of various cellular processes such as proliferation, migration, differentiation, inflammation, and their utility as biomarkers of pulmonary fibrosis beginning to be explored (Li et al., (2014), Int. J. Mol. Med., 33:1554-1562; Makiguchi et al., (2016), Respir. Res., 17:110; Yang et al., (2015), Gene, 562:138-144). miRs are small noncoding RNA molecules (20-22 nucleotides) that post-transcriptionally modulate gene expression by regulating the stability and/or translation of target mRNAs by binding to the 3′untranslated region of mRNAs (Baek et al., (2008), Nature, 455:64-71). Recent studies have reported an aberrant expression of miRs in the context of IPF. Indeed, Lui et al. found an upregulation of miR-21-5p in the lungs of patients with IPF and its pro-fibrotic properties by promoting the activity of TGF-β1 in pulmonary fibroblasts (Liu et al., (2010), J. Exp. Med., 207:1589-1597). On the other hand, other studies reported that the levels of miR-200c-5p (Yang et al., (2012), Am. J. Pathol., 180:484-493), miR-26a-5p (Liang et al., (2014), Mol. Ther., 22:1122-1133), miR-29-5p (Xiao et al., (2012), Mol. Ther., 20:1251-1260) and Let-7d-5p (Pandit et al., (2010), Am. J. Respir. Crit. Care Med., 182:220-229) were reduced in the lungs of mice with experimental pulmonary fibrosis and/or in lungs of patients with IPF, and their overexpression attenuated experimental pulmonary fibrosis in mice. However, the role of miRs in the pathogenesis of lung fibrosis is still being elucidated.

Exosomes are small vesicles (30-200 nm) that are secreted into biofluids (e.g. blood (Caby et al., (2005), Int. Immunol., 17:879-887) and bronchoalveolar lavage fluid (BAL) (Admyre et al., (2003), Eur. Respir. J., 22:578-583)) by several cell types, including alveolar epithelial cells, fibroblasts and inflammatory cells, and contain numerous bioactive molecules such as nucleic acids (including miRs), proteins and lipids (Mathivanan et al., (2012), Nucleic Acids Res., 40:D1241—D1244). Their content varies according to the state of their parental cells and is thus reflective of the cellular context (Njock and Fish, (2017), Trends Endocrinol. Metab., 28:237-246). Several studies reported that exosomes regulate various signaling pathways, including inflammatory and angiogenic pathways, by transferring miRs from a donor to a recipient cells (Bovy et al., (2015), Oncotarget, 6:10253-10266; Njock et al., (2015), Blood, 125:3202-3212; Tkach and Thery, (2016), Cell, 164:1226-1232). Exosomes and miRs appear to contribute to pulmonary fibrosis, as the size, quantity, content and function of exosomes vary with inflammation and epithelial damage (Martin-Medina et al., (2018), Am. J. Respir. Crit. Care Med., 198:1527-1538; Njock and Fish, (2017), Trends Endocrinol. Metab., 28:237-246), and several miRs are known to modulate fibrosis pathways (Jiang et al., (2010), FEBS J., 277:2015-2021). Very few studies have reported the role of exosomal miRs on pathogenesis of pulmonary fibrosis. Recently, Tan et al. have showed that exosomes from amnion epithelial cells are able to prevent and reverse lung fibrosis in bleomycin-treated mouse by targeting inflammatory and fibrotic pathways (Tan et al., (2018), Stem Cells Transl. Med., 7:180-196). In another study, Martin-Medina et al. have reported that extracellular vesicles from BAL of IPF patients contribute to disease pathogenesis by increasing lung fibroblast proliferation via a WNTSA-Dependent Manner (Martin-Medina et al., (2018), Am. J. Respir. Crit. Care Med., 198:1527-1538). In the art, contradicting reports have been published on both the role of miR142-3p in fibrosis and on its effect on pro-inflammatory genes. For example, miR-142-3-p has been demonstrated to inhibit apoptosis and inflammation induced by bleomycin through down-regulation of genes involved in inflammation such as Cox-2 in mouse lung epithelial cells in Guo et al.((2017), Braz. J. Med. Biol. Res., 50(7): e5974). In contrast, other reports (such as Wang et al. (2016), Arthritis Res Ther.; 18: 263) observed an upregulation of the inflammatory pathway due to miR-142-3p treatment. There is thus no consensus in the art on the role that miR-142-3p plays in fibrosis or on inflammation associated herewith.

WO2016054094 discloses a pharmaceutical composition comprising an effective amount of miRNA and an exosome isolated from a body fluid of a non-diseased subject to treat fibrotic disease including pulmonary fibrosis.

WO2013078283 discloses a method for treating a human subject with IPF comprising administering to the subject an oligonucleotide that modulates the activity or the level of expression of different miRNAs including mir-142, mir-let-7d, mir-26a or mir-33a.

The inventors have identified 3 miRs presenting an aberrant expression in sputum-derived exosomes from IPF patients compared to healthy subjects (miR-142-3p, miR-33a-5p, let-7d-5p) (Njock et al., (2019), Thorax, 74:309-312), but nothing has been elucidated regarding the role of such miRNA in the IPF disease progression.

There is thus a clear need to elucidate the exact role of miR-142-3p on fibrosis, how miR-142-3p influences the inflammation pathway, and to provide therapeutic strategies that exploit any beneficial anti-fibrotic effects of miR-142-3p while mitigating any unwanted side effects.

### SUMMARY OF THE INVENTION

The inventors have unravelled the cellular origin and the impact of miR-142-3p on pulmonary fibrosis progression. They found that levels of exosomal miR-142-3p are positively correlated with sputum macrophages of IPF patients. Furthermore, the inventors discovered that extracellular vesicles comprising miR-142-3p contain potent anti-fibrotic properties due in part to the transfer of miR-142-3p to alveolar epithelial cells and lung fibroblasts. Through extensive experiments, as illustrated in the example section herein, the inventors show that extracellular vesicles comprising miR-142-3p exert protective effects against fibro-inflammatory diseases via the delivery of miR-142-3p, and demonstrate that extracellular vesicles comprising miR-142-3p are suitable to be used as therapeutic agent to treat such diseases. The inventors have thus found that pharmaceutical formulations that comprise extracellular vesicles that are characterized by the presence of miR-142-3p in their contents have a beneficial effect on the group of fibro-inflammatory diseases. In the art, contradicting reports have been published on the role of miR-142-3p on fibrosis, and even on the influence that miR-142-3p has on pro-inflammatory genes. By employing more sophisticated experimental conditions compared to those in the art, the inventors provide comparative data showing that administration of miR-142-3p as such in fact significantly upregulates pro-inflammatory genes. This induction of inflammatory genes can be mitigated, and even actively counteracted, by packaging miR-142-3p in extracellular vesicles such as exosomes. A comparative study using highly relevant experimental conditions has not been performed in the art, which is likely causing the contradicting reports on miR-142-3p scattered throughout the art. The inventors hence show that the pro-inflammatory effect of miR-142-3p may be effectively avoided by packaging miR-142-3p in extracellular vesicles such as exosomes while retaining potent anti-fibrotic activity, which is an unexpected finding. Thus, the anti-fibrotic properties of miR-142-3p packaged in extracellular vesicles are independently of any effect on inflammatory genes. This finding is particularly relevant for the treatment of fibro-inflammatory diseases including but not limited to IPF and systemic fibrosis.

The invention can be captured in the following numbered statements:

Statement 1. A pharmaceutical formulation comprising extracellular vesicles produced by an in vitro cellular expression system, wherein the extracellular vesicles comprise an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1).

Statement 2. The pharmaceutical formulation according to statement 1, wherein the nucleic acid sequence has preferably at least 85% identity to the nucleic acid sequence of microRNA miR-142-3p (SEQ ID NO: 1), more preferably at least 90% identity to the nucleic acid sequence of microRNA miR-142-3p (SEQ ID NO: 1), more preferably at least 95% identity to the nucleic acid sequence of microRNA miR-142-3p (SEQ ID NO: 1).

Statement 3. The pharmaceutical formulation according to statement 1 or 2, wherein the extracellular vesicles are exosomes.

Statement 4. The pharmaceutical formulation according to any one of statements 1 to 3, wherein the extracellular vesicles are derived from macrophages or fibroblasts.

Statement 5. The pharmaceutical formulation according to any one of statements 1 to 4, wherein the pharmaceutical formulation is configured for intratracheal, intrabronchial, subcutaneous, transdermic, intravenous, aerosolized, nasal, intramucosal, intra-articular, or sublingual administration.

Statement 6. The pharmaceutical formulation according to any one of statements 1 to 5, wherein the content of the extracellular vesicles further comprises at least one additional component.

Statement 7. A process for obtaining a pharmaceutical formulation according to any one of statements 1 to 6 comprising following steps:
culturing an in vitro cellular expression system;
isolating extracellular vesicles from the cellular expression system; and
introducing an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1), into the extracellular vesicles.

Statement 8. A process for obtaining a pharmaceutical formulation according to any one of statements 1 to 6 comprising following steps:
culturing an in vitro cellular expression system wherein an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1) is present in the cellular expression system; and
isolating extracellular vesicles from the cellular expression system.

Statement 9. The process according to statement 8, wherein the oligonucleotide is an exogenous oligonucleotide.

Statement 10. The process according to any one of statements 7 to 9, further comprising a step wherein an agent is provided to the cellular expression system that increases the activity or the level of microRNA miR-142-3p.

Statement 11. The process according to any one of statements 7 to 10 wherein the in vitro cellular expression system is composed of macrophages or fibroblasts cells.

Statement 12. The process according to any one of statements 7 to 11, wherein the cellular expression system is cultured in hypoxic and/or acidic conditions.

Statement 13. The process according to any one of statements 7 to 12, wherein the miR-142-3p and/or extracellular vesicle components are transcribed from recombinant DNA.

Statement 14. The process according to any one of statements 7 to 13, wherein the nucleic acid sequence of miR-142-3p is comprised in a carrier RNA.

Statement 15. A pharmaceutical formulation as defined in any one of statements 1 to 6, for use as a medicament.

Statement 16. A pharmaceutical formulation as defined in any one of statements 1 to 6, for use in the treatment or prevention of fibro-inflammatory diseases.

Statement 17. The pharmaceutical formulation for use according to statement 16, wherein the fibro-inflammatory disease is idiopathic pulmonary fibrosis, systemic fibrosis, non specific interstitial lung disease, connective tissue disease, sarcoidosis, fibrosing sarcoidosis, chronic hypersensitivity pneumonitis, asbestosis, dermatomyositis, polymyositis, antisynthetase syndrome, cryptogenic organizing pneumonia, pulmonary fibrosis with auto-immune features, combined pulmonary fibrosis and emphysema, rheumatoid arthritis, arthrosis, crohn disease as well as ulcero-hemorragic rectocolitis.

Statement 18. The pharmaceutical formulation for use according to any one of statements 15 to 17 wherein at least one additional component is combined with the pharmaceutical formulation prior to administration.

Statement 19. The pharmaceutical formulation for use according to any one of statements 15 to 18, wherein the pharmaceutical formulation is administered at multiple points in time.

The invention can be further described according to the following numbered statements:

Statement 20. A pharmaceutical formulation comprising extracellular vesicles produced by an in vitro cellular expression system, wherein the extracellular vesicles comprise an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1), for use in the treatment or prevention of fibro-inflammatory diseases.

Statement 21. The pharmaceutical formulation for use according to statement 20, wherein said sequence has preferably at least 85% identity to the nucleic acid sequence of microRNA miR-142-3p (SEQ ID NO: 1), more preferably at least 90% identity to the nucleic acid sequence of microRNA miR-142-3p (SEQ ID NO: 1), more preferably at least 95% identity to the nucleic acid sequence of microRNA miR-142-3p (SEQ ID NO: 1).

Statement 22. The pharmaceutical formulation for use according to statement 20 or 21, wherein the extracellular vesicles are selected from the group of extracellular vesicles consisting of: microvesicles (MVs), exosomes, oncosomes, and apoptotic bodies, preferably wherein said extracellular vesicles are exosomes.

Statement 23. The pharmaceutical formulation for use according to any one of statements 20 to 22, wherein the extracellular vesicles are derived from monocytes, macrophages or fibroblasts, preferably THP-1 monocytes, THP-1 macrophages or HLF fibroblasts.

Statement 24. The pharmaceutical formulation for use according to any one of statements 20 to 23, wherein the fibro-inflammatory disease is selected from the group of fibro-inflammatory diseases consisting of: idiopathic pulmonary fibrosis, systemic fibrosis, non specific interstitial lung disease, connective tissue disease, sarcoidosis, fibrosing sarcoidosis, chronic hypersensitivity pneumonitis, asbestosis, dermatomyositis, polymyositis, antisynthetase syndrome, cryptogenic organizing pneumonia, pulmonary fibrosis with auto-immune features, combined pulmonary fibrosis and emphysema, rheumatoid arthritis, arthrosis, crohn disease as well as ulcero-hemorragic rectocolitis, preferably wherein the fibro-inflammatory disease is idiopathic pulmonary fibrosis or systemic fibrosis.

Statement 25. The pharmaceutical formulation for use according to any one of statements 20 to 24, wherein the pharmaceutical formulation is configured for intratracheal, intrabronchial, subcutaneous, transdermic, intravenous, aerosolized, nasal, intramucosal, intra-articular, or sublingual administration.

Statement 26. The pharmaceutical formulation for use according to any one of statements 20 to 25, wherein the expression levels of one or more pro-fibrotic genes selected from the group consisting of: COL1A1, COL3A3, and TGF-β1 is decreased by at least 15%, more preferably at least 25%, at least 50%, at least 75%, at least 90% in a subject treated with the pharmaceutical formulation when compared to a subject not receiving any anti-fibrotic treatment.

Statement 27. The pharmaceutical formulation for use according to any one of statements 20 to 26, wherein the expression level of one or more pro-inflammatory genes selected from the group consisting of: TNFα, IL-1β, and COX2 is decreased by at least 10%, preferably at least 20%, more preferably at least 30%, more preferably at least 50% in a subject treated with said pharmaceutical formulation when compared to a subject not treated with said pharmaceutical formulation.

Statement 28. The pharmaceutical formulation for use according to any one of statements 20 to 27, wherein at least one additional pharmaceutical active ingredient is combined with the pharmaceutical formulation prior to administration.

Statement 29. The pharmaceutical formulation for use according to statement 28, wherein said at least one additional pharmaceutical active ingredient is present in the extracellular vesicles of the pharmaceutical formulation.

Statement 30. A process for obtaining a pharmaceutical formulation according to any one of statements 20 to 29 comprising following steps:
culturing an in vitro cellular expression system;
isolating extracellular vesicles from the cellular expression system; and
introducing an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1), into the extracellular vesicles.

Statement 31. A process for obtaining a pharmaceutical formulation according to any one of statements 20 to 29 comprising following steps:
culturing an in vitro cellular expression system wherein an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1) is present in the cellular expression system; and
isolating extracellular vesicles from the cellular expression system.

Statement 32. The process according to statement 31, wherein the oligonucleotide is an exogenous oligonucleotide.

Statement 33. The process according to any one of statements 30 to 32, further comprising a step wherein an agent is provided to the cellular expression system that increases the activity of microRNA miR-142-3p and/or the level of microRNA miR-142-3p that is present in the extracellular vesicle, preferably wherein said agent increases the activity and/or the level of miR-142-3p with present in the vesicle with at least 10%, preferably at least 25%, more preferably at least 50%, more preferably at least 75%.

Statement 34. The process according to any one of statements 30 to 33 wherein the in vitro cellular expression system comprises, consists essentially of, or consists of monocytes, macrophages or fibroblasts cells, preferably THP-1 monocytes, THP-1 macrophages or HLF primary fibroblasts.

In the following passages, different embodiments of the invention are described in more detail. Each embodiment so defined may be combined with any other embodiment or multiple embodiments unless the contrary is explicitly stated. In particular, any feature indicated as being preferred or advantageous may be combined with any other feature or features indicated as being preferred or advantageous.

### BRIEF DESCRIPTION OF THE FIGURES

FIG. 1 illustrates the characterization of exosome-like vesicles isolated from sputum and plasma.

FIG. 1A represents a schematic overview of experimental procedure. FIGS. 1B-C illustrate the representative size distribution of purified exosomes from sputum (B) and plasma (C) by dynamic light scattering analysis. A representative experiment of 3 replicates is shown. The average size distribution of the vesicles is 140±19.1 nm for sputum-exosomes and 51.8 nm (±10.2 nm) for plasma-exosomes. FIGS. 1D-E represent western blot of exosomal markers, CD63, CD81, and mitochondrial protein cytochrome c in lysates from sputum-exosomes (D) and plasma-exosomes (E). The exosome samples were collected from healthy subjects (HS).

FIG. 2 illustrates the elevated expression level of exosomal miR-142—in sputum and plasma of IPF patients compared to HS. FIG. 2A-F represent the expression of specific miRs in sputum- and plasma-exosomes (respectively the left and right graphs) of IPF patients compared to HS. 2A: miR-142-3p, 2B: miR-21-5p; 2C: miR-33a-5p; 2D: miR-200c-5p; 2E: Let-7d-5p; 2F: miR-26a-5p. ns: not significant; *: 0.05; **: 0.01; ***: 0.001. FIG. 2G represents the Venn diagram of the overlap of dysregulated miRNAs in sputum and plasma of IPF patients. MiR-142-3p is upregulated in both the sputum and plasma of IPF patients.

FIG. 3 illustrates that the increased miR-142-3p from IPF sputum-exosomes is positively associated with sputum macrophages. FIG. 3A represents the heat map of correlation matrix between dysregulated sputum miRs (miR-142-3p, miR-33a-5p), sputum cells (macrophages, neutrophils) and lung function (DLCO/VA (%)). * Correlation is significant at the 0.05 level (2-tailed). FIG. 3B represents the spearman correlation between exosomal miR-142-3p expression and % sputum macrophages from sputum supernatants of IPF patients.

FIG. 4 illustrates that miR-142-3p is enriched in human monocytes/macrophages. FIGS. 4A-D represent the expression of miR-33a-5p and miR-142-3p in A549 alveolar epithelial cell line (A), MRCS lung fibroblast cell line (B), THP-1 monocytes (C) and THP-1 macrophages (D). FIG. 4E represents the relative expression of miR-33a-5p and miR-142-3p in A549, MRCS, THP-1 monocyte/macrophage cell lines.

FIG. 5 illustrates the biological pathways associated to sputum dysregulated miRs from IPF patients. (A) In silico analysis to predict functional role of IPF-related miRs (miR-142-3p, miR-33a-5p, Let7d-5p) with mirPath V3 (Tarbase V7 database). (B) predicted modulation of the TGF-B signaling pathways by the three IPF-related miRs.

FIG. 6 illustrates that miR-142-3p blocks fibrotic response by targeting TGFβ-R1. FIGS. 6A-G represents that miR-142-3p overexpression represses RNA and protein levels of TGFβ-R1 in A549 cells (A, D, E) and MRCS cells (B, F, G) as assessed by qRT-PCR and western blot analysis, and in HLF primary fibroblasts (C) as assessed by qRT-PCR. FIGS. 6H-J represent that miR-142-3p overexpression suppresses the induction of pro-fibrotic genes (COL1A1, COL3A1, TGF-β1) in response to TGF-β stimulation in A549 (H), MRCS (I) cells, and HLF primary fibroblast (J) as assessed by qRT-PCR. Throughout the graphs of the figure, the left bar indicates the control mimic condition, and the right bar the miR-142-3p mimic condition. The latter is further differentiated from the control mimic condition by filled squares indicative for the experimental results.

FIG. 7 illustrates macrophage-exosomes transfer of miR-142-3p to recipient A549 and MRCS cells. FIG. 7A illustrates a schematic representation of the experiment setup. FIG. 7B illustrates representative size distribution of purified THP1 macrophage-derived exosomes by dynamic light scattering analysis. A representative experiment of 3 replicates is shown. The average size distribution of the vesicles is 143.4±4.4 nm. FIG. 7C represents western blot analysis of exosomal markers, CD81, CD9, synthenin and mitochondrial protein cytochrome c in THP1 macrophage-derived exosomes and cell lysates from THP1 cells. FIGS. 7D-E represent the expression of IPF-related miRs in A549 alveolar epithelial cell line (D) and in MRCS lung fibroblast cell line (E) after incubation with (filled bars) or without (white bars) exosomes from THP1 macrophages. A significant difference in miR-142-3p can be observed in both cell lines.

FIG. 8 illustrates that macrophage-derived exosomes present anti-fibrotic properties. FIGS. 8A-B represent that macrophages exosomes were added on A549 (A) and MRCS (B) cells. FIGS. 8C-F represent that macrophage-exosomes represses TGFβ-R1 in A549 cells (C, D, E) and in RNA levels in MRCS fibroblasts (F). FIGS. 8G-H represent that macrophage-exosomes suppresses the induction of pro-fibrotic genes (COL1A1, COL3A1, TGF-β1) in response to TGF-β1 in A549 cells (G) and MRCS fibroblasts (H). Throughout the graphs of the figure, the left bar is indicative for the condition without macrophage-derived exosomes, and the right bar is indicative for the condition with macrophage-derived exosomes. In the latter, the experimental results are indicated with filled squares.

FIG. 9 illustrates the delivery of cel-miR-67 in vivo. cel-miR-67 was introduced in endothelial-derived exosomes by electroporation. 1.10⁵ 4 T1 cells were injected in the flank of Balb/c mice. 2 μg of the exosomes was injected subcutaneously in the peritumoral region in Blab/c mice every 2 days over a total period of 18 days. The level of cel-miR-67 in the tumor was assessed by qPCR.

FIG. 10 illustrates that direct transfection of A549 cells with miR-142-3p mimic (25 nM) increases the level of pro-inflammatory genes (TNF-α, IL-1β and COX2). In contrast, when exosomes of macrophages RAW298 transfected with miR-142-3p (25 nM) were added to A549 cells, the level of pro inflammatory genes is reduced compared to all other conditions. Conditions for each inflammatory gene from left to right: Ctrl mimic 25 nM, miR-142-3p mimic 25 nM, EXO+ctrl mimic 25 nM, EXO+miR-142-3p 25 nM.

FIG. 11 illustrates that macrophage-derived exosomes loaded with miR-142-3p inhibitor blocks the anti-fibrotic properties of exosomes. FIG. 11A illustrates that THP-1 macrophages were transfected with miR-142-3p inhibitor and that the miR-142-3p depleted macrophage exosomes collected were then added to A549 cells. FIG. 11B demonstrates that miR-142-3p is depleted in A549 cells (right bar, filled squares indicate experimental replicates). FIGS. 11C-F represent that miR-142-3p depleted macrophage-exosomes suppresses the blockade of pro-fibrotic genes (COL1A1, COL3A1, TGF-β1) and TGFβ-R1 expression in A549 cells. Left condition: PBS; middle condition: control inhibitor-exosome (not affecting the miR-142-3p content); right condition: miR-142-3p inhibitor exosomes.

FIG. 12 illustrates that macrophages loaded with miR-142 present anti-fibrotic activities. FIG. 12A represents the outline of the experiment. FIG. 12B shows the level of miR-142-3p in A549 cells incubated with exosomes transfected with miR-Ctrl mimic (i.e. cel-miR-67) or miR142-3p mimic, and that miR-142-3p mimic exosomes repress the expression of TGFβ-R1 in A549 cells (C) and pro-fibrotic genes (COL1A1, COL3A1, TGF-β1) (D-F). In the graphs of FIG. 12C-F, the left sample consistently is the PBS control condition, the middle sample is the ctrl mimic exosome condition, and the right sample is the miR-142-3p mimic exosome condition.

FIG. 13 illustrates the decrease of lung fibrosis in mouse having received miR-142-3p exosomes compared to mouse having received PBS. FIG. 13 shows a decrease of lung fibrosis in mice having received miR-142-3p exosomes (FIG. 13C-D) compared to mice receiving only bleomycin (FIG. 13A-B). Mice were injected intratracheally with bleomycin on day 1 (A-D) or PBS (E-F). On day 7 and 14, panels (C-D) illustrate mice injected intratracheally with miR-142-3p mimic loaded exosomes. Magnifications: 1.5× (panel A, C, E) and 20× (panel B, D, F). Quantification of the area of fibrosis (relative to the total lung area) is shown in panel G.

### DETAILED DESCRIPTION OF THE INVENTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +1-20% or less, preferably +/−15% or less, more preferably +/−10% or less, even more preferably +/−5% or less, most preferably +/−1%, and even most preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any >3, >4, >5, >6 or >7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects of the invention are defined in more detail. Each aspect so defined may be combined with any other aspect or aspects unless clearly indicated to the contrary.

In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may refer to general aspects of the invention. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The current invention uses conventional techniques of tissue culture, immunology, molecular biology, microbiology, cell biology and recombinant DNA, which are known to a person skilled in the art (e.g., Sambrook, Fritsch and Maniatis, (1989), Molecular Cloning: A Laboratory Manual, 2^{nd} edition; F. M. Ausubel, et al., (1987), MacPherson et al. Current Protocols in Molecular Biology; Methods in Enzymology: PCR 2: A Practical Approach, (1995); Harlow and Lane, (1988), Antibodies, A Laboratory Manual; Harlow and Lane, (1999), Using Antibodies, A Laboratory Manual; R. I. Freshney, (1987), Animal Cell Culture).

As used herein, a “control” such as a “control sample” may be used to correlate and compare the results obtained in the methods described herein from a test sample, in particular a sample from a subject to be diagnosed, herein also referred to simply as “sample”. A control level can be determined by measuring a sufficiently large number of control samples. A control can also represent an average value gathered from a number of tests or results. A person skilled in the art will recognize that controls may be designed for assessment of any number of parameters. For example, a control may be devised to compare therapeutic benefit based on pharmacological data (e.g., half-life) or therapeutic measures (e.g., comparison of side effects). The person skilled in the art will further understand which standard controls are most appropriate in any given situation and is able to analyse data based on comparisons to standard control values. Standard controls are also valuable for determining the significance (e.g. statistical significance) of data. For example, if values for a given parameter are characterized by a wide variation in standard controls, variation in test samples will not be considered as significant.

The following detailed description is not to be taken in a limiting sense. The scope of the present invention is defined by the appended claims.

The present inventors have found that extracellular vesicles such as exosomes containing microRNA miR-142-3p can be produced and used to treat fibro-inflammatory diseases such as IPF and systemic sclerosis. Remarkably, when using miR-142-3p packaged in extracellular vesicles such as exosomes, the observation could be made that miR-142-3p is still able to counter fibrosis without induction of key inflammatory genes. Hence, the anti-fibrotic properties of miR-142-3p are surprisingly independent of its impact on inflammation. This is in stark contrast to using unpackaged miR-142-3p, which causes a profound upregulation of these inflammatory genes, as evidenced in the examples section.

Hence, the present invention provides a pharmaceutical formulation comprising extracellular vesicles produced by an in vitro cellular expression system, wherein the extracellular vesicles comprise an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1). The terms “pharmaceutical formulation”, “pharmaceutical composition”, or “pharmaceutical preparation” may be used interchangeably herein. Likewise, the terms “formulation”, “composition”, or “preparation” may be used interchangeably herein. The term “extracellular vesicles” or “EVs”, as used herein, refers to all cell-derived vesicles secreted from cells. EVs are membrane surrounded structures released by cells that can transport cargo including but not limited to DNA, RNA, and proteins between cells. Different subtypes of EVs have been described and non-limiting examples include microvesicles (MVs), exosomes, oncosomes, and apoptotic bodies.

Microvesicles bud directly from the plasma membrane and have a size of about 100 nanometers (nm) to about 1 micrometer (m) in size. Exosomes are produced from the endosomal compartment and are typically formed by the fusion between multivesicular bodies and the plasma membrane, and have diameters ranging from about 40 nm to about 200 nm (El Andaloussi et al. (2013) Nat Rev Drug Discov, 12(5):347-57, Cocucci and Meldolesi (2015) Trends Cell Biol, 25(6):364-72). Dying cells release vesicular apoptotic bodies having a diameter of between about 50 nm and about 2 μm that can be more abundant than exosomes or MVs under specific conditions and can vary in content. Membrane protrusions can also give rise to large EVs termed oncosomes characterized by a diameter of about 1 μm to about 10 μm, which are produced primarily by malignant cells.

The terms “in vitro cellular expression system”, “expression system” or “cellular host system” may be used interchangeably herein and refer to any biological framework comprising cells that mediates expression of at least one population of EVs, such as but not limited to exosomes.

By the term “oligonucleotide” is meant oligomers and polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivative nucleotide bases. The backbone of nucleic acids can comprise sugars and phosphate groups, as can typically be found in RNA or DNA, and/or one or more modified or substituted sugars and/or one or more modified or substituted phosphate groups. Modifications of the phosphate groups or sugars may be introduced to improve stability, resistance to enzymatic degradation, or some other useful property. Furthermore, a person skilled in the art will appreciate that oligonucleotides may be (bio)chemically modified or contain non-natural or derivatives of naturally occurring nucleotides. By means of guidance and not limitation, the (bio)chemical modification or modifications may include introduction of labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). An oligonucleotide can be double-stranded, partly double stranded, or single-stranded. In embodiments where the oligonucleotide is single-stranded, the nucleic acid can be the sense strand or the antisense strand. In addition, oligonucleotides can be circular, linear, and/or may form specific secondary structures or structural motifs such as hairpins. The term oligonucleotide as used herein preferably encompasses pre-microRNA and microRNA.

The terms “microRNA”, “miRNA”, and “miR” may be used interchangeably. MicroRNAs (miRNAs) are small non-coding RNAs of about 20 to about 24 nucleotides that are abundant in many mammalian cell types. MicroRNAs play important roles in the regulation of target genes by binding to complementary regions of messenger transcripts to repress their translation or regulate degradation. MicroRNAs (miRNAs or miRs) are small RNA molecules that are transcribed from DNA into transcripts are called primary miRNAs (pri-miRNAs) that may contain the sequence encoding one or several microRNAs. This transcript is further processed into pre-miRNA that may adopt a secondary structure comprising a double stranded RNA stem and a single stranded RNA loop and further comprises the nucleotide sequence of the miRNA (and its complement sequence) in the double stranded RNA stem. Pre-microRNAs are processed into miRNAs. Processed microRNAs are single stranded oligonucleotides with a length of about 17 to about 25 nucleotides (nt) RNA molecules that become incorporated into the RNA-induced silencing complex (RISC). They play a role in regulating gene expression by binding to the 3′-untranslated region (UTR) of specific mRNAs. Typically, miRNAs down-regulate genes, so called target genes, by binding to the 3′ UTR of the respective targets. The binding is permissive for mismatches between the miRNA and the target gene and therefore does not need to be perfect across the whole mature miRNA sequence. Key for a miRNA to elicit its function is the so-called seed region (Lewis B P et al., (2003), Cell, 115:787-98). This seed region at the 5′ end of the mature miRNA consists of eight nucleotides. Various factors have been found to add to a strong seed binding. These include additional base pairs towards the 3′ end of the mature miRNA sequence.

A commonly used nomenclature system has been developed wherein microRNAs are annotated by “miR-xxx”, wherein x is a natural number from 0 to 9. MicroRNAs characterized by a very high degree of similarity that only differ in one or two nucleotides and may further be differentiated from each other by inclusion of an additional letter at the end of the name. Pre-miRNAs, pri-miRNAs and genes that lead to 100% identical mature miRNAs but situated at different genomic loci include an additional dash-number suffix. Optionally, the species of origin can be included in the annotation by a prefix such as hsa, which is indicative for a human (Homo sapiens) origin. Unless explicitly stated otherwise, the term “miR-142-3p” refers to human miR-142-3p (i.e. hsa-miR-142-3p). In case multiple mature microRNAs arise in approximately equal amount from opposite arms of the same pre-miRNA a “-3p” or “-5p” lowercase or uppercase suffix is added. Less recent methods to annotate miRNAs may indicate this with indicative sense and antisense abbreviations such as “s” and “as”. In case multiple miRNAs are produced from a pre-miRNA that have a distinct difference in their relative expression levels, an asterisk is commonly added as a suffix to the lowest expressed microRNA.

The current invention focusses on human miR-142-3p (hsa-miR-142-3P) characterized by the following canonical sequence:

(SEQ ID NO: 1)
5′ UGUAGUGUUUCCUACUUUAUGGA 3′

Human Pre-mir-142 has the following sequence:

(SEQ ID NO: 2)
5′GACAGUGCAGUCACCCAUAAAGUAGAAAGCACUACUAACAGCACUGG
AGGGUGUAGUGUUUCCUACUUUAUGGAUGAGUGUACUGUG 3′

A person skilled in the art is aware of tools and methodologies to verify sequence homology or sequence identity between different sequences of amino acids or nucleic acids. The percentage of identity between two sequences may show minor differences depending on the algorithm choice and parameters. The term “sequence identity” as used herein refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. A reference window is chosen and the “% identity” is calculated by determining the number of nucleotides that are identical between the sequences in the window, dividing the number of identical nucleotides by the number of nucleotides in the window and multiplying by 100. Unless indicated otherwise, the sequence identity is calculated over the whole length of the reference sequence. In the context of the current disclosure, a molecule or oligonucleotide having a sequence identity to a miR such as miR-142-3p is to be understood as an oligonucleotide having at least a local sequence identity to that miR, in this example miR-142-3p. A skilled person is aware that a local sequence identity of a certain percentage does not exclude the presence of additional sequences flanking the miR142-3p sequence. The term “flanking” indicates that said additional sequence is 3′ of the miR, 5′ of the miR, or that there is a first sequence 5′ of the miR and a sequence sequence identical or different to the first sequence on the 3′ of the miR. Therefore, a skilled person appreciates that oligonucleotide comprising a sequence having a certain percentage of identity to the nucleic acid sequence of microRNA miR-142-3p is to be interpreted as an oligonucleotide comprising a sequence that has that certain percentage of identity to miR142-3p.

In certain embodiments envisaged by the current invention, miRNA mimics may be used as alternative to the miRNA. miRNA mimics represent a class of molecules that can be used to imitate the gene silencing ability of one or more miRNAs. Thus, the term “microRNA mimic” refers to synthetic non-coding RNAs (i.e. the miRNA is not obtained by purification from a source of the endogenous miRNA) that are capable of entering and/or modulating the RNAi pathway and regulating gene expression. miRNA mimics can be designed as mature molecules (e.g., single stranded) or mimic precursors (e.g., pri- or pre-miRNAs). miRNA mimics can be comprised of nucleic acid (modified or modified nucleic acids) including oligonucleotides comprising, without limitation, RNA, modified RNA, DNA, modified DNA, locked nucleic acids, or 2′-0,4′-C-ethylene-bridged nucleic acids (ENA), or any combination of the above (including DNA-RNA hybrids). In certain embodiments, the miRNA mimic is a miR-142-3p mimic.

MicroRNA mimics with substantial identity of the mature microRNA should therefore conserve the activity of the naturally occurring mature miR. The term “microRNA mimic” is thus also indicative for a polynucleotide with a sequence that comprises at least 75% sequence identity compared to the reference sequence using a commonly used alignment algorithm using a standard set of parameters. Preferentially, the mutation will be located outside of the seed region of the microRNA.

As an illustrative example, miR-142-3p of the present invention has been produced from a synthetic RNA duplex (Ørom and Lund, (2007), Methods, 43:162-165). In summary, the sequence of miR-142-3p was modified with a phosphorylation at the 5′ end. The carrier strand (miR-142-reverse) was the complementary RNA sequence, which was also phosphorylated at the 5′ end and carried a UU 3′ overhang but with some mutations near the 3′ end to thermodynamically destabilize the strand and induce faster degradation. Oligonucleotides sense (hsa-miR-142-3p-S) and reverse (hsa-miR-142-3p-AS) were annealed and used as a duplex. The sequence and modifications introduced in the miR duplexes are

hsa-miR-142-3p-S:
(SEQ ID NO: 1)
5′ UGUAGUGUUUCCUACUUUAUGGA 3′,
hsa-miR-142-3p-AS:
(SEQ ID NO: 3)
5′ CAUAAAGUAGGCAACAUCUACCUU 3′.

“Functionally active fragments” as used herein is indicative for sequences comprising a functionally portion of the miRNA sequence. These sequences may be shorter than the original miRNA, but may also comprise additional sequences not occurring in the canonical sequence of the miRNA. Functionally active fragments retain at least a portion of the functionality of the miRNA. The functionally active fragments or mimics may furthermore be fused to additional oligonucleotide sequences to modulate the functionality of the miRNA.

In certain embodiments, single-stranded oligonucleotides, including those described and/or identified as microRNAs or miRs, may be used to serve as a template for the design of oligonucleotides of the invention. In certain embodiments, the cellular host system is an in vitro cultured population of cells. By means of guidance and not limitation, the cells used in the in vitro cellular expression system may be obtained from depositories such as the American Type Culture Collection (ATCC), the European Collection of Authenticated Cell Cultures (ECACC), Cellosaurus or any commercial vendor. In alternative embodiments, the cells used for the in vitro cellular expression system are obtained from isolating and/or immortalizing cells from model organisms, patients, or healthy subjects.

The EVs of the present invention may be obtained by isolating them from different types of cells, including the non-limiting examples of group of cell lines comprising endothelial cells, fibroblasts, mesenchymal stem cells, immune cells including macrophages, lymphocytes and neutrophils. Methods to produce, isolate, and characterize EVs are well known in the art (Thery et al., (2019), J. Extracell. Vesicles, 8:1535750). In certain embodiments, the EVs of the present invention are exosomes derived or isolated from human cells, preferably derived or isolated from A549 alveolar epithelial cells, MRCS lung fibroblast cells, THP-1 monocytes, RAW 264.7, THP-1 macrophages or HLF fibroblasts, preferably derived or isolated from MRCS lung fibroblast cells, THP-1 monocytes, or THP-1 macrophages. In certain embodiments, the EVs are derived or isolated from an in vitro cell culture comprising, consisting essentially of, or consisting of A549 alveolar epithelial cells, MRCS lung fibroblast cells, THP-1 monocytes, RAW 264.7, THP-1 macrophages, or HLF fibroblasts preferably derived or isolated from MRCS lung fibroblast cells, THP-1 monocytes, or THP-1 macrophages. In certain embodiments, the EVs of the present invention are isolated from a healthy subject, or a subject suspected to be in general good health, preferably a subject not diagnosed with or suspected of having any fibrotic disease, preferably not diagnosed with or suspected of having fibro-inflammatory diseases such as a pulmonary fibrosis disease, more preferably a subject not diagnosed with idiopathic pulmonary fibrosis.

EVs can be used immediately or stored for prolonged periods of time in temperatures ranging from about 4° C. to about −80° C. Examples of storage conditions and its impact on EV stability can be found in the prior art (Lee et al., (2016), Biotechnol. Bioprocess Eng., 21:299-304). Frozen EV can be stored in the presence of cryopreserving agents such as Trehalose (Bosch et al., (2016), Sci. Rep., 6:1-11). EVs, and by extension pharmaceutical formulations comprising EVs, can also be lyophilized to improve storage conditions or facilitate transport (Charoenviriyakul et al., (2018), Int. J. Pharm., 553:1-7).

The terms “subject” or “patient” are used interchangeably herein and refer to animals, preferably warm-blooded animals, more preferably vertebrates, and even more preferably mammals specifically including humans and non-human mammals, that have been the object of treatment, observation or experiment. The term “mammal” includes any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes. Particularly preferred are human subjects, including both genders and all age categories thereof. Non-human animal subjects may also include prenatal forms of animals, such as, e.g., embryos or foetuses. Human subjects may also include foetuses, but by preference not embryos. In certain embodiments, the subject is a subject diagnosed with, or suspected of having a fibrotic disease, preferably a fibro-inflammatory disease, more preferably (idiopathic) pulmonary fibrosis or systemic fibrosis. In certain embodiments, the miRNA can be a synthetic oligonucleotide.

The term “synthetic oligonucleotide” as used herein refers to oligonucleotide sequences which may be synthesized de novo using any of a number of procedures well known in the art. These procedures include chemical synthesis methods (Laikhter A. and Linse K., 2014, The chemical synthesis of oligonucleotides, Biosynthesis) or enzymatic synthesis methods (Perkel J., 2019, The race for enzymatic DNA synthesis heats up, Nature).

In certain embodiments, the nucleic acid sequence has preferably at least about 85% identity to the nucleic acid sequence of microRNA miR-142-3p (SEQ ID NO: 1), more preferably at least about 90% identity to the nucleic acid sequence of microRNA miR-142-3p (SEQ ID NO: 1), more preferably at least about 95% identity to the nucleic acid sequence of microRNA miR-142-3p (SEQ ID NO: 1). In further embodiments, the nucleic acid sequence has preferably at least about 96% identity to the nucleic acid sequence of microRNA miR-142-3p (SEQ ID NO: 1), preferably at least about 97% identity to the nucleic acid sequence of microRNA miR-142-3p (SEQ ID NO: 1), preferably at least about 98% identity to the nucleic acid sequence of microRNA miR-142-3p (SEQ ID NO: 1), preferably at least about 99% identity to the nucleic acid sequence of microRNA miR-142-3p (SEQ ID NO: 1). In certain embodiments, the nucleic acid sequence is identical to the canonical microRNA miR-142-3p sequence provided in SEQ ID NO: 1.

In certain embodiments, miR-142-3p is the most abundant miR present in the extracellular vesicles.

In certain embodiments, the extracellular vesicles comprised in the pharmaceutical composition are selected from the group of extracellular vesicles consisting of: microvesicles, exosomes, oncosomes, and apoptotic bodies. In further embodiments, the extracellular vesicles comprised in the pharmaceutical composition are microvesicles. In certain embodiments, the extracellular vesicles comprised in the pharmaceutical formulation are exosomes.

The term “exosomes” as used herein refers to EVs that are produced in the endosomal compartment of most eukaryotic cells. Exosomes are commonly defined as lipid bilayer enclosed membrane extracellular vesicles that are released from cells upon fusion of an intermediate endocytic compartment, the multivesicular body (MVB), with the plasma membrane. Exosomes are present in tissues and can also be found in biological fluids including blood, urine, sputum, and cerebrospinal fluid. They are also released in vitro by cultured cells into culture-conditioned medium. Exosomes from different organisms or produced by different celltypes may differ in their protein to lipid ratio, luminal content and sedimentation characteristics. Subtypes of exosomes have been reported and despite their relative homogenous characteristics, they mediate a broad spectrum of effects on recipient cells. Exosomes may be engulfed by acceptor cells or the exosome membrane may fuse directly with the host plasma membrane. Cellular markers to identify exosomes include CD9, CD 81, CD63, synthenin, ALIX and TSG101. Microvesicles size can be estimated by techniques provinding images at high resolution like eletronic microcopy or techniques that estimate biophysical features like dynamic ligth scattering. Various methods for the isolation of exosomes from biological fluids have been developed and include techniques involving centrifugation, chromatography, filtration, polymer-based precipitation and immunological separation. These methodologies are available in the state of the art (Yakimchuk K, (2015), Materials and methods, 5:1450). Furthermore, methods to separate subtypes of exosomes have been reported wherein a difference in migration speed in a sucrose gradient may be part of the method, as sucrose migration speed is influenced by density and size.

In certain embodiments, the extracellular vesicles comprised in the pharmaceutical formulation are a subset of exosomes characterized by additional defining traits such as optionally a different migration speed in a sucrose gradient. In certain embodiments, the exosomes are modified by chemical substances, proteases, or enzymes prior to introduction to the pharmaceutical formulation. In certain embodiments, the exosomes are concentrated between isolation and introduction to the pharmaceutical formulation. In further embodiments, the exosomes have a human origin. In certain embodiments, the exosomes comprise non-naturally occurring components.

In certain embodiments, the extracellular vesicles in the pharmaceutical formulation are derived from monocytes or fibroblasts. In certain embodiments, the extracellular vesicles in the pharmaceutical formulation are derived from macrophages or fibroblasts. In further embodiments, the extracellular vesicles are derived from macrophages or fibroblasts derived from a human subject. In certain embodiments, the extracellular vesicles are derived from an in vitro cell culture of THP-1 monocytes, THP-1 macrophages, or HLF fibroblasts.

The term “macrophage” refers to a certain type of a white blood cell part of normally occurring as part of an immune system. A skilled person is aware that monocytes are generally accepted as the precursor cells of macrophages and dendritic cells. Macrophages are capable of engulfing and digesting cellular debris, both endogenous and exogenous substances, microbes, or other cell types including cancer cells by phagocytosis. Depending on place and form, macrophages are commonly given alternative names including the following non-limiting examples: adipose tissue macrophages, monocytes, Kupffer cells, Sinus histiocytes, alveolar macrophages, tissue macrophages, microglia, Hofbauer cells, Intraglomerular mesangial cells, osteoclasts, epithelioid cells, red pulp macrophages, peritoneal macrophages, LysoMac. Activated macrophages can be stratified in two main groups, M1 and M2. M1 are commonly regarded as killer macrophages while M2 macrophages are often referred to as repair macrophages. There have been a large amount of markers reported in the state of the art. By means of guidance and not limitation, following common markers are suitable to detect and identify macrophages of different anatomical locations: B7-1/CD80, B7-2/CD86, CCRS, CD11b/Integrin alpha M, CD1 1c, CD14, CD15/Lewis X, CD68/SR-D1, CD163, EMR1, F4/80, Fc gamma RIB/CD64b, Fc gamma RIII (CD16), Fc gamma RI/CD64, Fc gamma RII/CD32, Galectin-3, Galectin-3C, GITR Ligand/TNFSF18, HLA-DR, Integrin alpha L/CD11a, LAMP-2/CD107b, LILRB4/CD85k/ILT3, M-CSF R/CD115, MHC class II (I-A/I-E), Siglec-3/CD33, TLR2, TLR4. Furthermore, subtype specific markers have been reported and are known in the art.

The term “fibroblast” refers to a certain connective tissue cell type that are involved in producing the extracellular matrix and collagen, stroma for animal tissues. Additionally fibroblasts play a role in wound healing. Fibroblast marker proteins include vimentin, α-SMA, Desmin, FSP1, Discoidin-domain receptor 2, FAP, α1β1 integrin, prolyl 4-hydroxylase, pro-collagen 1α2, CD248, and VCAM-1.

A cell is said to be positive for (or to express or comprise expression of) a particular marker, when a skilled person will conclude the presence or evidence of a distinct signal, e.g., antibody-detectable or detection by reverse transcription polymerase chain reaction, for that marker when carrying out the appropriate measurement, compared to suitable controls. Where the method allows for quantitative assessment of the marker, positive cells may on average generate a signal that is significantly different from the control, e.g., but without limitation, at least 1.5-fold higher than such signal generated by control cells, e.g., at least 2-fold, at least 4-fold, at least 10-fold, at least 20-fold, at least 30-fold, at least 40-fold, at least 50-fold higher or even higher.

The expression of the above cell-specific markers can be detected using any suitable immunological technique known in the art, such as immuno-cytochemistry or affinity adsorption, Western blot analysis, FACS, ELISA, etc., or by any suitable biochemical assay of enzyme activity (e.g., for ALP), or by any suitable technique of measuring the quantity of the marker mRNA, e.g., Northern blot, semi-quantitative or quantitative RT-PCR, etc. Sequence data for markers listed in this disclosure are known in the art and can be obtained from public databases such as GenBank (http://www.ncbi.nlm.nih.gov/).

In certain embodiments, the extracellular vesicles in the pharmaceutical formulation may be derived from activated macrophages. In certain embodiments, the extracellular vesicles in the pharmaceutical formulation may be derived from genomically modified macrophages, fibroblasts, or both. In certain embodiments, the EVs in the pharmaceutical formulation may be derived from monocytes, macrophages and/or fibroblasts specific for a disease condition including but not limited to human lung (primary) (HLF) fibroblasts, THP-1 monocytes, or THP-1 macrophages. In certain embodiments, the EVs may further comprise marker proteins expressed in macrophages and/or fibroblasts. In certain embodiments, the macrophages and/or fibroblasts may be derived from a patient. In further embodiments, the patient is diagnosed with a fibro-inflammatory disease, preferably a (idiopathic) pulmonary fibrosis disease. In further embodiments, the macrophages and/or fibroblasts are cultured in optimized culture conditions for EV production. In certain embodiments, the macrophages and/or fibroblasts have been transiently exposed to certain culture conditions to increase EV production. In further embodiments, the macrophages and/or fibroblasts were generated from stem cells. In a more specific embodiment, the stem cells were genomically modified stem cells. In certain embodiments, a non-naturally occurring genetic marker is present in the genome of the stem cells, macrophages, and/or fibroblasts that is indicative for high EV production. In certain embodiments, the marker is a protein expressed by the stem cells, macrophages, and/or fibroblasts.

In certain embodiments, the pharmaceutical formulation is configured for intratracheal, intrabronchial, subcutaneous, transdermic, intravenous, aerosolized, nasal, intramucosal, intra-articular, or sublingual administration.

In certain embodiments, the configured pharmaceutical formulation has improved storage characteristics, sensory appearance, or bioavailability.

The term “intratracheal” generally refers to a route of administration in which a pharmaceutically active ingredient (e.g. the extracellular vesicles as taught herein) is delivered into the trachea or windpipe. Alternatively, the method may be referred to as intratracheal instillation. Typically, instillation is performed either through inserting a needle or catheter down the mouth and throat, or through surgically exposing the trachea and penetrating it with a needle. In certain embodiments, additional anaesthetic drugs such as the non-limiting examples halothane, metaphane, enflurane may be co-introduced with the pharmaceutical formulation to the subject. In certain embodiments, the pharmaceutical formulation further comprises an additional component to reduce irritation.

The term “intrabronchial” generally refers to a route of administration in which a pharmaceutically active ingredient (e.g. the EVs as taught herein) is delivered into one or both of the bronchi. The delivery can be performed by means of injection. In certain embodiments, the intrabronchial administration may be performed by injecting the extracellular vesicles suspended in a suitable solvent such as a salt solution into one or both of the bronchi. In certain embodiments, the pharmaceutical formulation configured for intrabronchial administration is also suitable for intratracheal administration.

The term “subcutaneous” generally refers to a route of administration in which a pharmaceutically active ingredient (e.g. the EVs as taught herein) is delivered or administered into the subcutis, the layer of skin directly below the dermis and epidermis, collectively referred to as the cutis. In certain embodiments the pharmaceutical formulation comprises an adjuvant to facilitate or increase the efficiency of subcutaneous delivery such as aluminium-based adjuvant or substitutes such as calcium phosphate nanoparticles.

The term “transdermic” generally refers to a route of administration in which a pharmaceutically active ingredient (e.g. the EVs as taught herein) is delivered or administered by a dermal application. In certain embodiments, the pharmaceutical formulation is configured for a specific transdermic administration entity, i.e. a means suitable for, or adapted to allow transdermic administration. By means of guidance and not limitation, this may be a spray, a lotion, a cream, an ointment, a gel, a gum, a bandage, a dermal patch, or a plaster.

The term “intraveinous” or “intravenous” generally refers to a route of administration in which a pharmaceutically active ingredient (e.g. the EVs or exosomes comprising miR-142-3p as taught herein) is delivered or administered into the vein.

The term “aerosolized” generally refers to a route of administration in which a pharmaceutically active ingredient (e.g. the EVs as taught herein) is delivered or administered with inhaled therapy (with dry powder, pMDI, aerosolization or any device leading to an airway deposition). In certain embodiments, additional components are introduced to the pharmaceutical formulation which beneficially impact aerosolization. In certain embodiments, additional components are mixed with the pharmaceutical formulation during the aerosolization.

In certain embodiments, the pharmaceutical formulation may be configured to facilitate nasal administration.

The term “intra mucosal” generally refers to a route of administration in which a pharmaceutically active ingredient (e.g. the EVs as taught herein) is delivered or administered to mucus, also known as airway surface liquid.

The term “intra-articular” generally refers to a route of administration in which a pharmaceutically active ingredient (e.g. the EVs a taught herein) is delivered or administrated by entry into a joint.

The term “sublingual” generally refers to a route of administration in which a pharmaceutically active ingredient (e.g. the regulatory macrophages as taught herein) is delivered or administered by applying it under the lingua. In certain embodiments, additional components may be added to the pharmaceutical formulation that modulate the taste of the formulation. In further embodiments, these may be selected from the group of noncaloric, noncarcinogenic sweeteners, e.g. saccharin, aspartame, acesulfame K, or cyclamate.

In certain embodiments, the additional component is an non-active pharmaceutical ingredient. “Non-active pharmaceutical ingredients” or “inactive ingredients” are components of a drug formulation that do not increase or affect the therapeutic action of the one or more active ingredients. These are commonly referred to as excipients. In certain embodiments, the excipient may be an active pharmaceutical ingredient excipient, binder excipient, carrier excipient, co-processed excipient, coating system excipient, controlled release excipient, diluent excipient, disintegrant excipient, dry powder inhalation excipient, effervescent system excipient, emulsifier excipient, lipid excipient, lubricant excipient, modified release excipient, penetration enhancer excipient, permeation enhancer excipient, pH modifier excipient, plasticizer excipient, preservative excipient, preservative excipient, solubilizer excipient, solvent excipient, sustained release excipient, sweetener excipient, taste making excipient, thickener excipient, viscosity modifier excipient, filler excipient, compaction excipient, dry granulation excipient, hot melt extrusion excipient, wet granulation excipient, rapid release agent excipient, increased bioavailability excipient, dispersion excipient, solubility enhancement excipient, stabilizer excipient, capsule filling excipient. The use of such media and agents for pharmaceutical active substances is well known in the art. Such materials should be non-toxic and should not interfere with the activity of the pharmaceutically active ingredients. In certain embodiments, more than one excipient from the same group is added to the pharmaceutical formulation. In further embodiments, more than one excipient wherein the different excipients belong to different groups are added. In certain embodiments, the excipients may fulfill more than one function.

Furthermore, the formulation may comprise pharmaceutically acceptable auxiliary substances as required to approximate physiological conditions, such as pH adjusting and buffering agents, preservatives, complexing agents, tonicity adjusting agents, wetting agents and the like, for example, sodium acetate, sodium lactate, sodium phosphate, sodium hydroxide, hydrogen chloride, benzyl alcohol, parabens, EDTA, sodium oleate, sodium chloride, potassium chloride, calcium chloride, sorbitan monolaurate, triethanolamine oleate, etc.

The term “release” as used herein refers to the release of a component of the pharmaceutical formulation, such as an active pharmaceutical ingredient over a defined period of time. The meaning of “release of a pharmaceutical active ingredient” is known to a person skilled in the art and is indicative for displacement of said active ingredient from the interior of a confined pharmaceutical dosage form to the exterior of said dosage form. The release of said pharmaceutical active ingredient may be initiated or occurring by means of a so-call passive release mechanism, such as but by no means limited to diffusion, or by means of an active release mechanism, such as but not limited to mechanical pressure. Depending on inter alia the excipients further contained in the pharmaceutical compositions, the release may be immediate, delayed, or sustained.

In alternative embodiments, the additional component is an active pharmaceutical ingredient. In certain embodiments, the additional component belongs to the group of analgesics. As treatment of fibro-inflammatory diseases is envisioned, in certain embodiments the additional active ingredient may be classified as an anti-inflammatory agent that reduces or prevents inflammation. In certain embodiments the additional active ingredient may be a nonsteroidal anti-inflammatory drug (NSAID). In alternative embodiments, the anti-inflammatory drug may be an immune selective anti-inflammatory drug or derivative (ImSAID). In alternative embodiments, the anti-inflammatory drug may be a selective glucocorticoid receptor agonist (SEGRA). In alternative embodiments, the anti-inflammatory drug may be a resolvin or a protectin. In alternative embodiments, the anti-inflammatory drug may be be a anti-inflammatory biological. In alternative embodiments, the anti-inflammatory drug is a natural anti-inflammatory agent. In certain embodiments, the additional active pharmaceutical ingredient is contained in the EV. Inclusion of an anti-inflammatory ingredient different than the miR-142-3p in an extracellular vesicle such as an exosome provides the advantage that the anti-inflammatory properties of miR-142-3p when comprised in exosomes may be further exploited. The additional anti-inflammatory ingredient may either act independently of the anti-inflammatory effect of miR-142-3p exosomes, or in a synergistic manner. Since unpackaged miR-142-3p leads to a profound induction of inflammation as shown in detail in the examples section, using an anti-inflammatory ingredient in conjuction with unpackaged miR-142-3p would merely counteract the inflammation induction caused by the miR-142-3p, and not contribute to a further anti-inflammatory effect. By packaging the miR-142-3p in extracellular vesicles such as exosomes, further anti-inflammatory ingredients may be added to the pharmaceutical formulation, or even into said extracellular vesicles, that cater to the needs of an individual patient. Evidently, a skilled person appreciates that avoiding inflammation entails considerable clinical benefits for the patient.

The term “analgesics” as used herein refers to a group of drugs suitable to achieve relief from pain, i.e. analgesia.

“Pharmaceutical active ingredient” or “API” as referred to herein is to be interpreted according to the definition of the term by the World Health organization: a substance used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings.

In certain embodiments, the pharmaceutical formulation can be obtained by a process comprising following steps: culturing an in vitro cellular expression system, isolating extracellular vesicles from the cellular expression system, and introducing an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1), into the extracellular vesicles.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is intended any proliferation or division of cells.

“Isolating” refers to a process to obtain an isolated substance, in the present invention EVs, preferably exosomes. The term “isolated” as used herein refers to molecules or biological or cellular materials being substantially free from other materials, e.g., greater than 70%, or 80%, or 85%, or 90%, or 95%, or 98%. The term “isolated” is indicative for extracellular vesicles that are substantially free of other cellular material, viral material, or culture medium. The term “isolated” is also used herein to refer to cells, exosomes or microvesicles, miRNA, or tissues that are isolated from other cells, exosomes or microvesicles, miRNA, or tissues and products produced or isolated from such.

In certain embodiments, the in vitro cellular expression system is cultured in a fed-batch culture system. The term “fed-batch” refers to a culturing technique in biotechnological processes where one or more nutrients are fed to the bioreactor during cultivation and in which the products remain in the bioreactor or vessel. In further embodiments, the culture medium of the in vitro expression system is replenished with fresh medium in a continuous manner. In further embodiments, the medium is replenished when at least one parameter value for the culture is reached. By means of guidance and not limitation this parameter can be pH, absorbance, cell density, concentration of debris, cell viability. In certain embodiments, EVs produced by the in vitro cellular expression system are extracted from the system in a continuous manner. In alternative embodiments, the culture is maintained in a certain cell density range. In certain embodiments, the cells are cultured in serum free conditions. In further embodiments, alternative components are added to the culture medium with similar functions to serum. Alternatives for fetal bovine serum have been published in the art (Gstraunthaler G., (2003), Alternatives to the use of fetal bovine serum: serum-free cell culture, 20(4):275-81). In certain embodiments, the composition of the culture medium may be varied depending on the specific stage of extracellular vesicle production.

The term “culture medium” refers to a substance used to support maintenance and/or proliferation and/or differentiation of living cells. The culture media can be a water-based media which includes a combination of ingredients such as salts, nutrients, minerals, vitamins, amino acids, nucleic acids, proteins such as cytokines, growth factors and hormones, all of which may contribute to cell survival, proliferation, and/or differentiation. In certain embodiments, the used culture medium may be a combination of at least two different culture media. In certain embodiments, conditioned medium may be used in the process. Conditioned medium refers to medium wherein cells have been cultured for a period in time in before using the medium for the process described in the current invention, and wherein first cells have released certain components such as proteins, cytokines, chemicals, etc. to the medium. Typically said cultured cells are removed from the medium by means such as but not included to filtration prior before further using the conditioned medium to aid in the maintenance, proliferation, and/or differentiation of the further cells. Methods to generate conditioned medium are known in the art. In certain embodiments, concentrated conditioned medium may be used in the process.

In certain embodiments, the production of extracellular vesicles can be initiated by addition of a certain component to the culture medium. In alternative embodiments, extracellular vesicles are produced continuously. In certain embodiments, the cells are grown in hypoxic conditions and/or incubated in medium with a pH indicative for an acidic environment in order to increase the yield of the extracellular vesicles. In the current invention, known methods in the art developed for the isolation of extracellular vesicles are envisaged and encompass techniques not limited to those associated with beads, columns, filters, precipitation and antibodies.

In certain embodiments, an isolation method specific for exosomes is used. In certain embodiments, the isolation may be facilitated by incorporation of an additional marker on the surface of the extracellular vesicles or inside the extracellular vesicles. In further embodiments, the process further comprises a step to verify introduction of microRNA miR-142-3p into the EVs. In certain embodiments, the process further comprises an additional step to separate miR-142-3p containing EVs from EVs that do not contain miR-142-3p. In certain embodiments, additional copies of microRNA miR-142-3p are added to the EVs already containing miR-142-3p. In certain embodiments, additional microRNAs are introduced to the miR-142-3p containing EVs. In certain embodiments, at least one component of the EVs is overexpressed.

“Overexpressed” or “overexpression” as used herein refer to technologies known in the art to encompass increasing the expression of a nucleic acid or a protein to a level greater than the EV or exosome naturally, i.e. physiologically, contains. It is intended that the term encompasses overexpression of endogenous nucleic acids and proteins, as well as heterologous expression of nucleic acids and proteins. Hence, a skilled person appreciates that both endogenous genes may be upregulated to overexpress one or more gene products, or that (artificial) nucleic acids such as expression vectors or artificial chromosomes may be introduced to a cellular system of overexpress gene products or fragments thereof that do not naturally occur in said cell type, said tissue type, or even said organism.

In certain embodiments, the in vitro cellular expression system is composed of macrophages or fibroblast cells. In certain embodiments, the in vitro cellular expression system comprises, consists essentially of, or consists of monocytes, macrophages, and/or fibroblasts, preferably primary fibroblasts.

In certain embodiments, the cells of the in vitro cellular expression system are derived from macrophages or fibroblast cells. In certain embodiments, the in vitro cellular expression system comprises, consists essentially of, or consists of THP-1 monocytes, THP-1 macrophages, RAW 264.7 macrophages, or HLF primary fibroblasts. In certain embodiments, cell types from different origin, lineage, or passage number are used in the same in vitro cellular expression system. In certain embodiments, the in vitro cellular expression system comprises macrophages or fibroblasts adapted for suspension culturing conditions. Methods and reagents suitable to culture macrophages or fibroblasts have been reported throughout the state of the art and are commercially available.

In certain embodiments, the pharmaceutical formulation can be obtained by a process comprising following steps: culturing an in vitro cellular expression system wherein an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1) is present in the cellular expression system; and isolating extracellular vesicles from the cellular expression system. In further embodiments, the isolation step for the extravesicular vesicles is specific for exosomes. In alternative further embodiments, the isolation step allows differentiation between exosome and other extracellular vesicles such as apoptotic bodies. In yet further alternative embodiments, a first isolation step is performed to isolate any extracellular vesicles and a second isolation step is performed on the obtained extracellular vesicles to further isolate exosomes from said extracellular vesicles.

In certain embodiments, the cells overexpress miR-142-3p compared to the natural occurring corresponding cell type. In certain embodiments, the average expression level of miR-142-3p in said cells is at least 1.5 fold, preferably at least 2.5 fold, more preferably at least 5 fold, more preferably at least 10 fold elevated when compared to the naturally occurring (i.e. non-modified or non-treated) cells of the same cell type. In certain embodiments, miR-142-3p is expressed in the cellular expression system only after introduction of an oligonucleotide sequence comprising the coding sequence for miR-142-3p in the cells. Hence, a cell type not expressing miR142-3p may also be contemplated as a cellular expression or production system for extracellular vesicles comprising miR-142-3p, or preferably exosomes comprising miR-142-3p. Introduction of a oligonucleotide into a cell is a well-known concept to one skilled in the art, and methods have been published to achieve said introduction. As used herein, “introduction” refers to exogenous addition of an oligonucleotide into a cell. Examples of such techniques include, but are not limited to transfection, lentiviral infection, nucleofection, electroporation or transduction. In some embodiments, the exogenous addition process comprises introducing a vector comprising the gene of interest. The vector may be a DNA plasmid delivered via non-viral methods or via viral methods. In certain embodiments, a miR-142-3p expression cassette is transiently expressed in the cells. Methods to generate expression cassettes are known to a person in the art.

The term “expression cassette” as used herein refers to part of DNA, which may be part of a vector, comprising, consisting essentially of, or consisting of a gene or portion of DNA and a regulatory sequence including but not limited to a promoter sequence. By using different regulatory sequences such as distinct promoter sequences or enhancer sequences, the level of expression may be regulated.

In certain embodiments, the oligonucleotide is an exogenous oligonucleotide. In certain embodiments, the exogenous oligonucleotide sequence or segment may be circular or linear, double-stranded or single-stranded. Generally, the exogenous sequence is in the form of chimeric DNA, such as plasmid DNA or a vector that can also contain coding regions flanked by control sequences that promote the expression of the recombinant DNA present in the transformed cell. In certain embodiments, the exogenous oligonucleotide is a segment of a DNA sequence coding for a miRNA, pri-miRNA, or pre-miRNA. In certain embodiments, the exogenous oligonucleotide further encodes for at least one additional mRNA, pri-miRNA, or pre-miRNA. Preferably, the at least one additional mRNA, pri-MRNA, or pre-miRNA may be that of (hsa-)miR-33a-5p, or (hsa-)let-7d-5p, or any combination thereof.

“Control sequences” or “regulatory sequences” as referred to herein is any sequence of nucleic acid molecules which is capable of increasing or decreasing the expression of specific genes. This regulation may be imposed by either influencing transcription rates, translation rates in case of protein coding sequences, or by modification of the stability of the sequence. In further embodiments, the polynucleotide sequence comprises regulatory elements such as but not limited to the following: promoter, enhancer, selection marker, origin of replication, linker sequences, polyA sequence, degradation sequence.

In certain embodiments, the oligonucleotide may comprise the miRNA-142-3p sequence in the form of a carrier RNA such as the pri-miR or the pre-miR. In alternative embodiments, the miR-142-3p expression cassette is permanently integrated into the genome of the cells. In further embodiments, the activity of the miR-142-3p expression cassette can be regulated by an exogenously introduced component. In yet further embodiments, multiple miR-142-3p expression cassettes are introduced in a cell. In further embodiments, insertion of the expression cassette into the host cell genome can be verified by conferred resistance of the miR-142-3p expression cassette containing cells to certain selective pressure such as antibiotic treatment. In yet further embodiments, the miR-142-3p containing cells are characterized by expression of a fluorescent marker. In further embodiments, the miR-142-3p may be introduced as such in the cellular expression system. In certain embodiments, further components to be included in the extracellular vesicles, preferably exosomes are introduced by any means disclosed herein in the cellular expression system. In further embodiments, these components may be but are by no means limited to (hsa-)miR-33a-5p, and/or (hsa-)let-7d-5p. Alternatively, these components may have as a function upon incorporation in the membrane of the extracellular vesicles to improve targeting, binding, or delivering the cargo of said extracellular vesicles to target cells in general or specific (sub-)populations of target cells.

In certain embodiments, the cells are treated with a component that increases the export and/or level and/or activity of miR-142-3p in EVs. In certain embodiments, the component (which may interchangeably be indicated by the term “agent” is provided to the cellular expression system that increases the activity of microRNA miR-142-3p and/or the level of microRNA miR-142-3p that is present in the extracellular vesicle, preferably wherein said agent increases the activity and/or the level of miR-142-3p with present in the vesicle with at least 10%, preferably at least 25%, more preferably at least 50%, more preferably at least 75%. In certain embodiments, the activity of the miR-142-3p that is present in the extracellular vesicles is increased by at least 1.5-fold, preferably at least 2-fold, more preferably at least 5 fold, or even at least 10-fold when compared to miR-142-3p in extracellular vesicles not comprising said component. In certain embodiments, the level, i.e. concentration of the miR-142-3p that is present in the extracellular vesicles is increased by at least 1.5-fold, preferably at least 2-fold, more preferably at least 5 fold, or even at least 10-fold when compared to miR-142-3p in extracellular vesicles not comprising said component.

In further embodiments, this component may be an anti-fibrotic agent such as but not limited to pirfenidone, nintedanib, crizotinib, rapamycin, anti-IL6 or anti-IL7. In yet further embodiments, the component may be an oligonucleotide. In certain embodiments, the component may be substantially present in the produced EVs. In certain embodiments, more than one component is added to the culture. In embodiments where the component is a protein, the protein may be transcribed and/or translated in the in vitro cellular expression system. Alternatively, said protein may be introduced in a recombinant form to the in vitro cellular expression system.

Also intended is the use of the pharmaceutical formulation comprising extracellular vesicles part of an in vitro cellular expression system, wherein the content of the extracellular vesicles comprises an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1) as defined in any one of the embodiments described herein as a medicament.

Also intended is the use of the pharmaceutical formulation comprising extracellular vesicles part of an in vitro cellular expression system, wherein the content of the extracellular vesicles comprises an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1) as defined in any one of the embodiments in the treatment or prevention of fibrotic diseases.

Also intended is the use of the pharmaceutical formulation comprising extracellular vesicles part of an in vitro cellular expression system, wherein the content of the extracellular vesicles comprises an oligonucleotide comprising at least 75% identity to the nucleic acid sequence of microRNA miR-142-3p (SEQ ID NO: 1), or a functionally active fragment of the nucleic acid sequence of microRNA miR-142-3p (SEQ ID NO: 1) as defined in any one of the embodiments in the treatment or prevention of fibro-inflammatory diseases.

In certain embodiments, the pharmaceutical formulation may be configured to contain a prophylactic effective amount. In alternative embodiments, the pharmaceutical formulation may be configured to contain a therapeutically effective amount for a subject in need of a treatment.

As used herein, a phrase such as “a subject in need of treatment” includes subjects that would benefit from treatment of a given condition, particularly a fibro-inflammatory disease. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed fibro-inflammatory disease, as well as prophylactic or preventive measures, wherein the aim is to prevent or lessen the chances of incidence of an undesired affliction, such as to prevent occurrence, development and progression of a fibro-inflammatory disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician.

The formulations and methods as taught herein allow to administer a therapeutically effective amount of a pharmaceutical active ingredients in subjects having a fibro-inflammatory disease which will benefit from such treatment.

The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. In the context of the present invention a therapeutically effective dose means an amount of the extracellular vesicle as taught herein that when administered brings about a positive therapeutic response with respect to treatment of a patient with a fibrosing disease, or more specifically a fibro-inflammatory disease such as IPF or systemic fibrosis. Methods are known in the art for determining therapeutically and prophylactically effective doses of the regulatory macrophages or pharmaceutical composition as taught herein and depend on the nature of extracellular vesicle, the disease condition and severity, and the age, size and condition of the patient.

The term “fibro-inflammatory disease” refers to all fibrosing diseases that encompass a series of exceedingly different lesions that have a common denominator in the presence of varying amounts of newly formed fibrous tissue and different degrees of involvement of inflammatory processes. The biological nature and pathogenesis of the lesions are highly heterogeneous and vary from barely known diseases to iatrogenic disorders, autoimmune diseases, vasculitic forms, or neoplastic lesions. Hence, the term “fibrosing disease” refers to all diseases associated with an abnormal matrix deposition due to an inflammatory process, a lung aggression, an autonomous fibroblastic activity or any mechanism leading to abnormal fibroblastic activity.

In certain embodiments, administration of the extracellular vesicles comprising miR-142-3p, preferably exosomes comprising miR-142-3p induces a decrease in the expression levels of one or more pro-fibrotic genes in a subject. In further embodiments, the decrease in the expression levels of one or more pro-fibrotic genes in said subject is at least 10%, preferably at least 15%, at least 20%, at least 25%, at least 30%; at least 40%, at least 50%, at least 75%, at least 90%. In certain embodiments, the decrease in expression levels of one or more pro-fibrotic genes in a subject is between 10% to 95%, preferably between 15% and 95%, between 20% to 95%, between 25% to 90%, between 50% and 75%. In further embodiments, said pro-fibrotic genes are selected from COL1A1, COL3A1, and TGF-β1.

Remarkably, and in contrast to unpackaged miR-142-3p, the pharmaceutical formulation as described herein does not cause an induction of pro-inflammatory genes, providing a more elegant fibrosis treatment strategy. A skilled person is readily able to appreciate genes involved in inflammation from the art, as such genes have been published in detail (inter alia in Saradna, A., Do, D. C., Kumar, S., Fu, Q.-L. & Gao, P. Macrophage polarization and allergic asthma. Translational Research 191, 1-14 (2018)). In certain embodiments, the pharmaceutical formulation as described herein do not significantly upregulate pro-inflammatory genes. In alternative embodiments, the pharmaceutical formulation as described herein reduce the expression level of one or more pro-inflammatory genes with at least 10%, preferably at least 20%, more preferably at least 30%, more preferably at least 50% when compared to the expression level(s) of said inflammatory gene(s) in cells of a subject treated with unpackaged miR-142-3p. In certain or any embodiments described herein, the pro-inflammatory genes are TNFa, IL-10 and/or COX2. In certain embodiments, the expression level of each of the pro-inflammatory genes TNFa, IL-10, and COX2 is reduced by at least 10%, preferably by at least 20%, more preferably by at least 30%, more preferably by at least 50% when compared to the expression level(s) of said inflammatory gene(s) in cells of a subject treated with unpackaged miR-142-3p.

Hence, the extracellular vesicles comprising miR-142-3p, preferably exosomes comprising miR-142-3p, are able to reduce the expression level of one or more pro-fibrotic genes in a subject without further causing a clinically relevant alteration (such as a clinically relevant upregulation or statistically significant upregulation) of pro-inflammatory genes. In a certain embodiments, the extracellular vesicles comprising miR-142-3p, preferably exosomes comprising miR-142-3p, are able to reduce the expression level of COL1A1, COL3A1, and/or TGF-01 by at least 10%, preferably at least 15%, at least 20%, at least 25%, at least 30%, at least 40%, at least 50%, at least 75%, at least 90% while inhibiting the expression level of TNFa, IL-113, and/or COX2 by at least 10%, preferably at least 20%, preferably at least 30%, preferably at least 50%, preferably at least 60%, preferably at least 70% in (cells of) a patient diagnosed with, or suspected of having a fibro-inflammatory disease.

In certain embodiments, the fibro-inflammatory disease is idiopathic pulmonary fibrosis, systemic fibrosis, non specific interstitial lung disease, connective tissue disease, sarcoidosis, fibrosing sarcoidosis, chronic hypersensitivity pneumonitis, asbestosis, dermatomyositis, polymyositis, antisynthetase syndrome, cryptogenic organizing pneumonia, pulmonary fibrosis with auto-immune features, combined pulmonary fibrosis and emphysema, rheumatoid arthritis, arthrosis, crohn disease, or ulcero-hemorragic rectocolitis.

Diseases belonging to the group of fibro-inflammatory diseases have been described throughout the state of the art (Vaglio A, 2017, systemic fibroinflammatory disorders). It is understood that a skilled practitioner would be able to diagnose diseases belonging to the group of fibro-inflammatory diseases by diagnosis methods and diagnosis tools available in the art.

In certain embodiments, at least one additional component is combined with the pharmaceutical formulation prior to administration. In certain embodiments, the at least one additional component is an active pharmaceutical ingredient, such as but by no means limited to an anti-inflammatory agent. In further embodiments, the at least one active pharmaceutical ingredient is present in the extracellular vesicles of the pharmaceutical formulation.

In certain embodiments, the additional component is combined with the pharmaceutical formulation immediately prior to administration. In alternative embodiments where the pharmaceutical formulation is stored under a lyophilized condition, the additional component may be part of the solvent used to reconstitute the formulation. In certain embodiments, the amount of the additional component added to the pharmaceutical formulation is calculated based on certain patient parameters including but not limited to age, weight, gender, severity of the fibro-inflammatory disease condition, other known diseases of the patient. In certain embodiments, the pharmaceutical formulation may be used in combination with other formulations available for treatment of fibro-inflammatory diseases. In certain embodiments, the additional component may alter bioavailability by the non-limiting examples of altering biodistribution, solubility, and/or permeability. In certain embodiments, the additional component is an anti allergy agent.

In certain embodiments, the pharmaceutical formulation is administered at multiple points in time. In certain embodiments, the administered dose remains equal throughout the different administrations. In alternative embodiments, the dosage is increasing or decreasing in time. In certain embodiments, different pharmaceutical compositions may be used for the different administrations. In certain embodiments, the pharmaceutical composition of the formulation may be invariable, but the formulations used for the different administration points may be variable in the content of the EVs. In certain embodiments, the time interval between the multiple administrations may be constant. In alternative embodiments, the time interval between the multiple administrations may be increasing. In alternative embodiments, the time interval between the multiple administrations may be depending on certain disease parameters of the patient. In certain embodiments, the administration may be continuous. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1: Isolation and Characterization of Exosome-Like Vesicles from Sputum and Plasma of Ipf Patients

Patient Cohort.

Patients were recruited from the ambulatory care polyclinics of Liege University, Belgium. The protocol was approved by the Ethics Committee of CHU of Liege, and all subjects provided written consent before their enrolment (Belgian number: B707201422832, ref: 2014/302). The diagnosis of (definite) IPF was made according to the international recommendations of the ATS 1 using the respiratory function test, HRCT scan (probable UIP pattern), bronchoalveolar lavage (when available), as well as the clinical history of the patient. The patient characteristics for the sputum cohort are described in one of our previous studies (Njock et al., (2019), Thorax, 74:309-312) (Table 1). All other causes of interstitial lung disease (such pneumoconiosis, hypersensitivity pneumonitis, pneumonia associated with connective tissue disease or drug induced interstitial lung disease) were excluded. No patients showed any symptoms of a respiratory disease and all patients showed normal spirometric values with predicted FEV1>80% and FEV1/FVC ratio>70%. Patients treated with histone deacetylase inhibitors like theophylline were also excluded. None of the patients from cohort 1 have received specific anti-fibrotic therapy (nintedanib, pirfenidone). A second cohort was used to determine the levels of the microRNA of interest in plasma (Table2). In this plasma cohort, IPF patients where predominantly male (77%) with a mean age of 74 y. 2 IPF patients where active smokers whereas 6 of them where former smokers. They exhibit a restrictive respiratory pattern with a low FVC (74(18)%) with a reduced diffusing capacity of the lungs for carbon monoxide (DLCO). One of the patients was further treated with anti-fibrotic specific therapy and one patient with oral corticosteroids.

TABLE 1
Clinical characteristics of first cohort (cohort to
study the alteration of exosomal miRs in IPF sputum).

| Clinicopathological | HS | IPF |
|---|---|---|
| characteristics | nb = 14 | nb = 16 |
| Age, yrs | 62(4) | 73(9)** |
| Gender (M/F) | 5/9 | 10/5 |
| BMI, Kg/m² | 27(3) | 28(3) |
| Smokers (Never/ex/Current), n | 2/8/4 | 3/11/2 |
| pack-yr | 18(12) | 28(19) |
| FEV1 post-BD, % pred. | 104(13) | 72(13)*** |
| FVC post-BD, % pred. | 114(13) | 70(20)*** |
| FEV1/FVC post-BD, % | 78(5) | 81(10) |
| TLC, % pred. | nd | 62(12) |
| DLCO % pred. | nd | 37(20) |
| KCO % pred. | nd | 62(18) |
| OCS (yes/no) | 0/30 | 1/15 |

TABLE 2
Clinical characteristics of second cohort (cohort to
study the alteration of exosomal miRs in IPF plasma).

| | HS | IPF |
|---|---|---|
| Clinical characteristics | Nb = 9 | nb = 9 |
| Age, yrs | 67(4) | 74(9) |
| Gender (M/F) | 3/6 | 7/2 |
| BMI, Kg/m² | 27(4) | 28(4) |
| Smokers (NS/FS/S) | 0/7/2 | 1/6/2 |
| FEV1 post-BD, % pred. | 103(15) | 75(17)** |
| FVC post-BD, % pred. | 114(23) | 74(18)** |
| FEV1/FVC post-BD, % pred. | 74(7) | 78(10) |
| TLC, % pred. | nd | 69(17) |
| DLCO % pred. | nd | 43(17) |
| KCO % pred. | nd | 58(28) |
| Pirfenidone/Nintenadib yes/no | 0/9 | 1/8 |
| OCS yes/no | 0/9 | 1/8 |

Abbreviations: HS: Healthy subjects; IPF: idiopathic pulmonary fibrosis; BMI: Body mass index; FEV1: Forced expired volume in one second; FVC: Forced vital capacity; DLCO: Diffusing lung capacity with CO; KCO: DLCO/alveolar ventilation; OCS: oral corticosteroids. Data are presented as mean±SD or median (IQR), * p<0.05; ** p<0.01;***p<0.001.

Sputum Induction and Processing.

After premedication with 400 micrograms inhaled salbutamol, sputum was induced by inhalation of hypertonic (NaCl 5%) or isotonic (NaCl 0.9%) saline according to the FEV1 value (> or <than 65% predicted). Saline was combined with additional salbutamol delivered by an ultrasonic nebulizer (Ultra-Neb 2000; Devilbiss, Somerset, Pa., USA) with an output set at 0.9 ml/min. Each subject inhaled the aerosol for three consecutive periods of 5 min for a total of 15 min. The whole sputum was collected in a plastic container, weighed, and homogenized by adding three volumes of phosphate-buffered saline (PBS), vortexed for 30 s, and centrifuged at 800 g for 10 min at 4° C. Supernatant was separated from cell pellet and stored at −80° C. For safety reasons, FEV1 was monitored throughout the induction and induction was stopped if FEV1 fell by more than 20% from baseline.

Isolation of Microvesicles (MV) and Exosomes from Sputum and Plasma.

Exosomes from sputum and plasma were isolated by a standard ultracentrifugation protocol. First, the biofluids were resuspended in PBS and precleared by centrifugation at 400 g for 5 minutes, prior to centrifugation at 2,000 g for 20 min at 4° C. To isolate microvesicles, the supernatants was centrifuged at 20,000 g for 120 min at 4° C. The supernatant was discarded and the MV pellet was resuspended in PBS or lysed with Qiazol and stored at −80° C. Then, the supernatant was passed through a 0.22-μm filter (Millipore). To isolate exosomes, the precleared supernatant of sputum or plasma was ultracentrifuged at 110,000 g for 120 minutes at 4° C., followed by washing of the exosome pellet with PBS at 110,000 g for 120 minutes at 4° C. (Optima XPN-80 Ultracentrifuge, Beckman Coulter). The supernatant was discarded and the exosome pellet was resuspended in PBS or lysed with Qiazol and stored at −80° C. Protein levels of the exosome preparations were measured using the BCA Protein Assay kit (Pierce) according to the manufacturer's instructions.

Dynamic Light Scattering.

Exosomes were suspended in PBS at a concentration of 50 μg/mL, and analyses were performed with a Zetasizer Nano ZS (Malvern Instruments, Ltd.). Intensity, volume and distribution data for each sample were collected on a continuous basis for 4 min in sets of 3.

Quantitative RT-PCR (qRT-PCR).

The miRNA PCR array data were validated by qRT-PCR. 50 ng RNA was reverse transcribed into cDNA using qScript miRNA cDNA Synthesis kit (Quanta Biosciences), and qRT-PCR was conducted in triplicate using Perfecta SYBR Green Super Mix (Quanta Biosciences). Thermal cycling was performed on an Applied Biosystems 7900 HT detection system (Applied Biosystems). The relative miRNA levels were normalized to 3 internal controls selected via Normfinder software, miR-222-3p, miR-191-5p and miR-16-5p, using the Delta-Delta Ct method. The primers were from Quanta Biosciences. The promising miRNAs were selected based on their fold change and p-value. miR-33a-5p and miR-142-3p present the highest increase with a small p-value (4.03 fold, p=0.0082; 3.03 fold, p=0.0291). Furthermore, the selected miRNAs were reported to be dysregulated in IPF context and play a major role in fibrosis progression.

Isolation and Characterization of Exosome-Like Vesicles from Sputum and Plasma of IPF Patients.

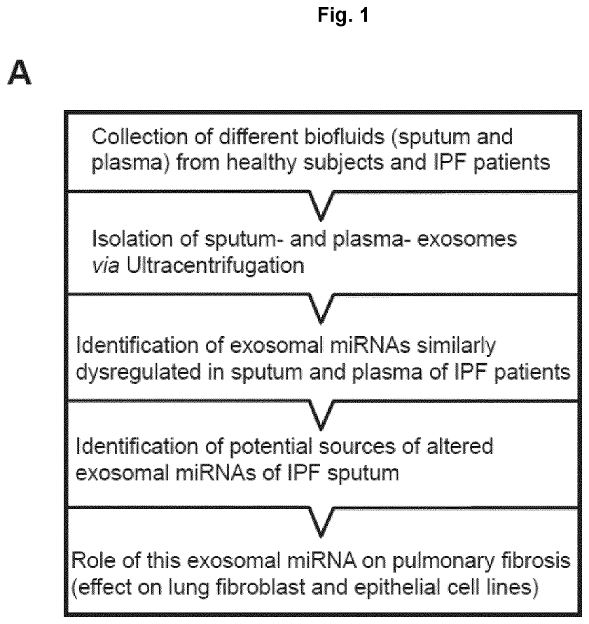

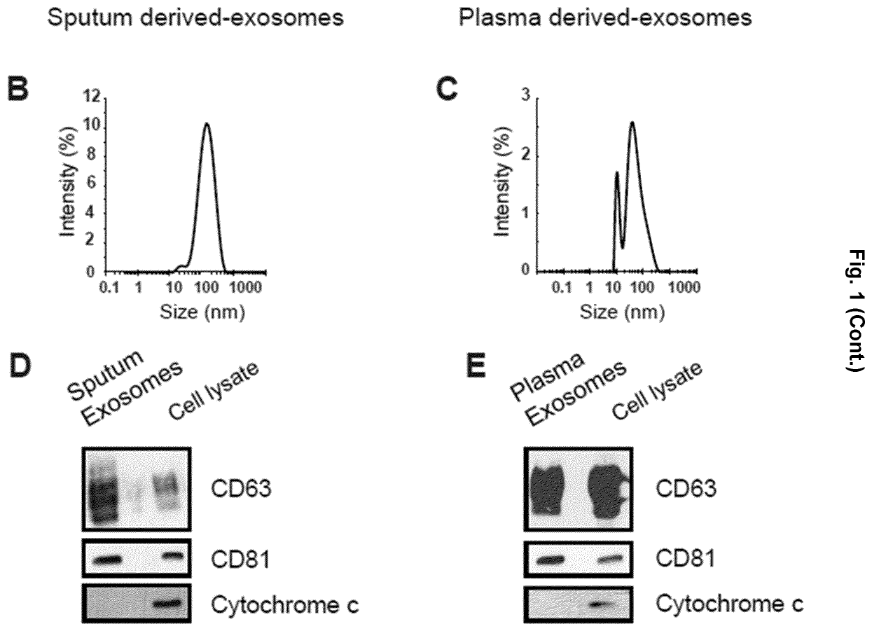

The exosomes from sputum and plasma of HS and IPF patients were isolated using a standard ultracentrifugation protocol (as previously, workflow depicted in FIG. 1A). The average size distribution of the vesicles purified from sputum and plasma was 140 nm (±19.1 nm) and 51.8 nm (±10.2 nm), respectively (FIG. 1.B-C), a typical exosome size distribution. Furthermore, the isolated vesicles present an enrichment of several exosomal markers, CD63 and CD81, and an absence of mitochondrial cytochrome C (FIG. 1.D-E), which confirms the purity of our sputum- and plasma-derived exosome preparation.

Western Blotting.

Soluble exosomes or cell lysates (5 μg) were resolved by SDS-PAGE (10-15%) and transferred to polyvinylidene fluoride membranes (Millipore). Blots were blocked 3 h with 5% milk in Tris-buffered saline (TBS) with 0.1% Tween-20, and blotted overnight with the following primary antibodies in blocking solution (directed to CD63 (#106228D, Invitrogen), CD81 (#10630D, Invitrogen), or cytochrome c (#556433, BD pharmingen). After 3 washes with TBS/0.1% Tween-20, membranes were incubated for 1 h at room temperature with an HRP-conjugated secondary antibody before being revealed with ECL substrate (Pierce Biotechnology).

Exosomal miR-142-.3p Levels is Increased in Induced Sputum and Plasma of IPF Patients.

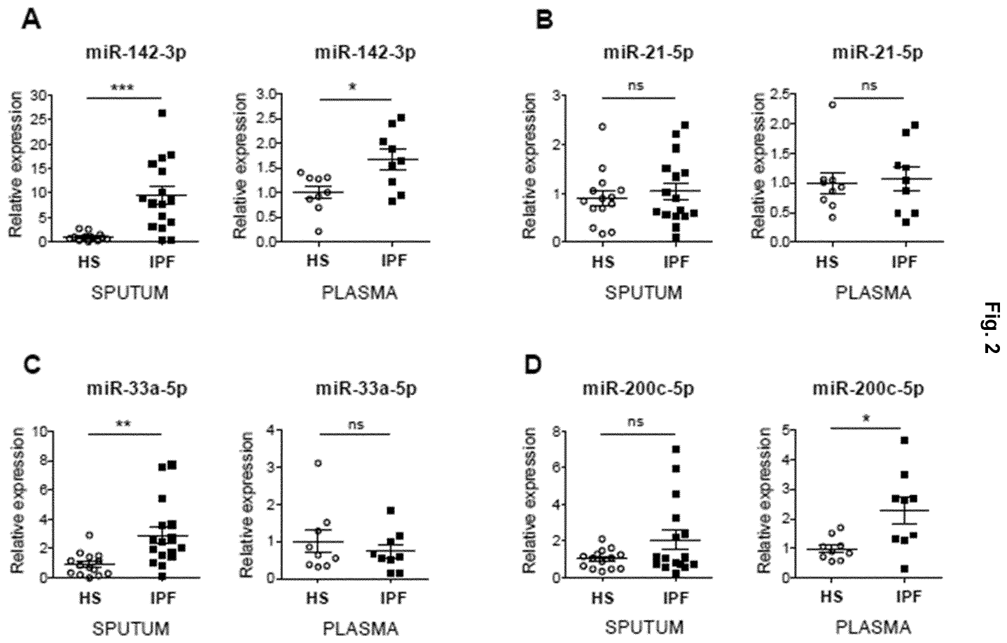

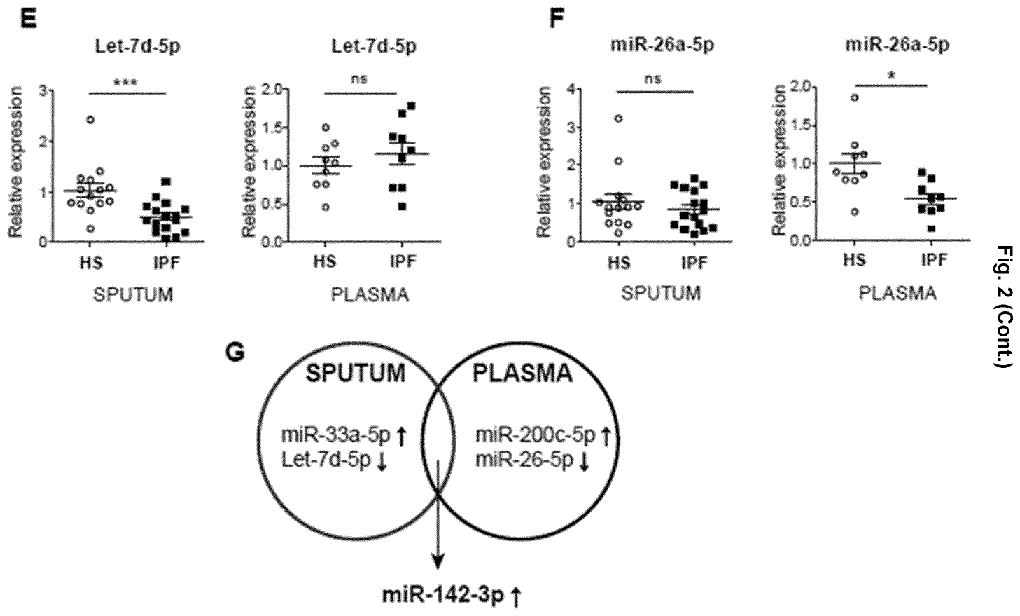

Previously, the inventors have identified a unique signature of three miRs (miR-142-3p, miR-33a-5p, let-7d-5p) as potential biomarkers for IPF from sputum-derived exosomes (Njock et al., (2019), Thorax, 74:309-312). For comparison purposes, the levels of sputum biomarkers in an alternative biofluid were determined. For this, plasma was deemed appropriate, as several studies have described the dysregulation of circulating miRs in plasma/serum from IPF patients, such as miR-21-5p, miR-200c-5p or miR-26a-5p. Changes of these miRs in sputum exosomes (16 IPF vs 14 HS) and plasma exosomes (11 IPF vs 12 HS) were assessed. Interestingly, only one miR, miR-142-3p, is dysregulated similarly in the exosomes from sputum and plasma of IPF patients compared to HS (FIG. 2.A, G), suggesting a major role in IPF pathophysiology. The alteration of this miR is more pronounced in the sputum (increase of 9.4 fold, p=0.0002) than the plasma (increase of 1.5 fold; p=0.021) (FIG. 2.A). The 2 other dysregulated miRs identified previously in the IPF sputum (miR-33a-5p, Let-7d-5p) are not affected in the IPF plasma (FIG. 2.C, E). Similarly, the levels of the 3 plasma dysregulated miRs, miR-21-5p, miR-200c-5p and miR-26a-5p, are not changing in the sputum from IPF patients (FIG. 2.B, D, F).

Exosomal miR-142-3p Expression is Positively Correlated with Sputum Macrophages of IPF Patients.

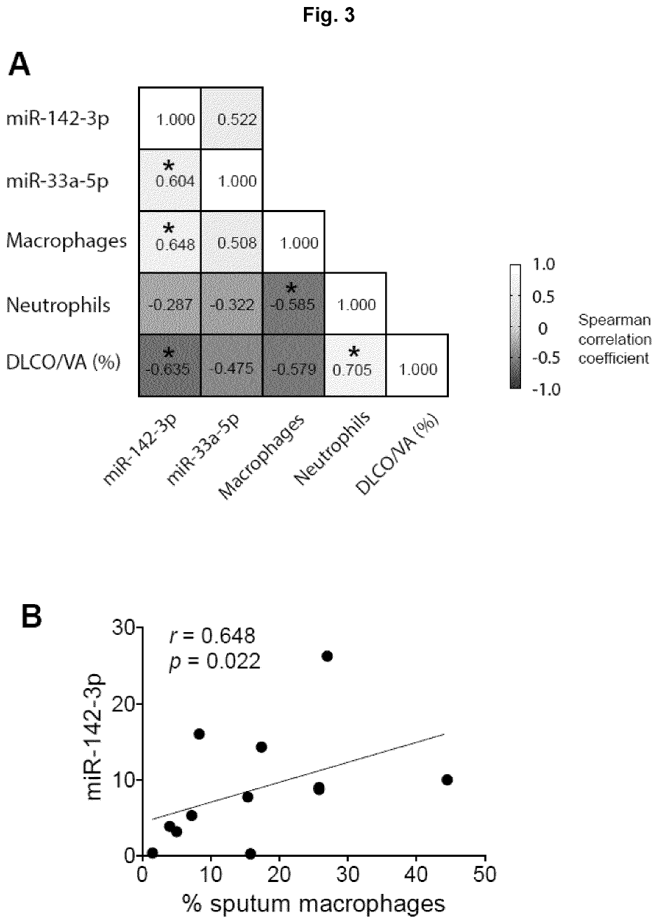

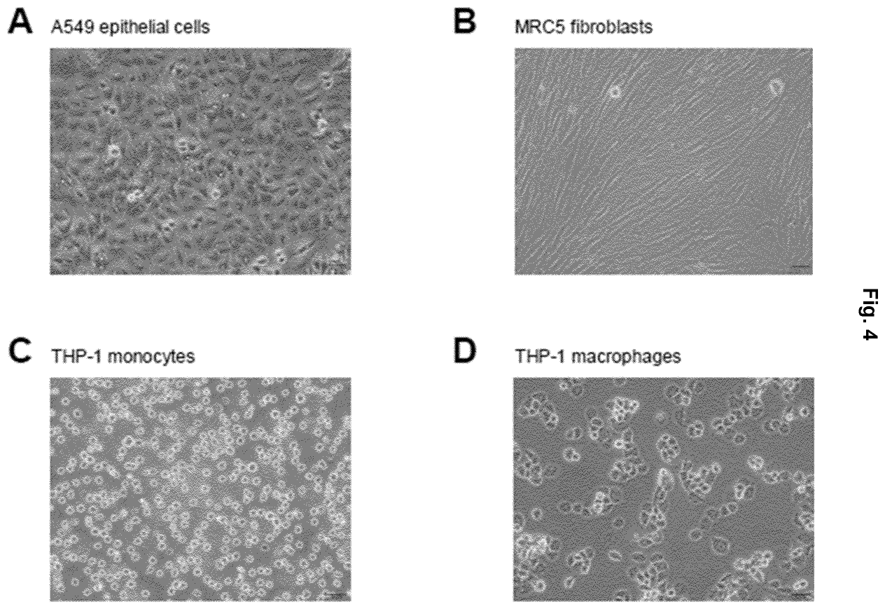

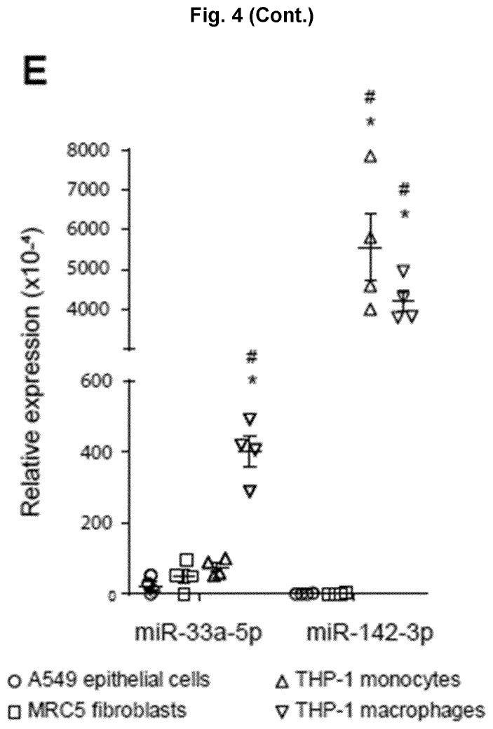

To investigate the cellular origin of miR-142-3p-enriched exosomes, the inventors performed correlation analysis between miR expression levels and sputum cells (FIG. 3.A). Expression of exosomal miR-142-3p was positively associated with the percentage of sputum macrophages (Spearman rho=0.648, p=0.022), suggesting that these immune cells are a major source of altered miR-142-3p in IPF sputum (FIG. 3.B). To confirm this, the levels of miR-142-3p in cell subsets present in sputum, alveolar epithelial cells (A549 cell line), monocytes (THP1 monocytic cell line) and macrophages (THP1 macrophages) (FIG. 4.A, C-D) were assessed. Lung fibroblast cells (MRCS cell line) were also included, as these cells could participate to the pool of sputum-derived exosomes (FIG. 4.B). Interestingly, miR-142-3p is highly enriched in THP1 monocytes/macrophages compared to A549 lung epithelial cells and MRCS fibroblasts (fold>2900, fold>3200, respectively, p<0.05; FIG. 4.E).

### Example 2. Biological Pathways Associated to Sputum Dysregulated miRNAs from IPF Patients

miRNA Target Prediction and Pathway Analysis.

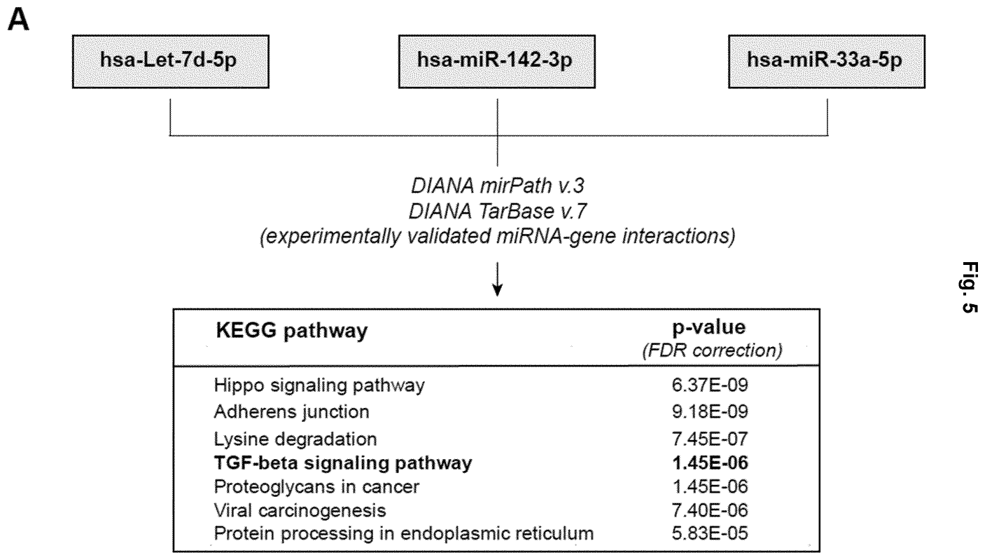

In order to elucidate the potential role of the three dysregulated miRNAs in IPF patients, the miRNAs were subjected to in silico analysis with mirPath v.3 using Tarbase v.7 database. The analysis revealed that these miRNAs are involved in the regulation of TGF-β signaling pathway (FDR=1.45E-6) (FIG. 5.A), a major process involved in the initiation and progression of pulmonary fibrosis by inducing epithelial cell/fibroblast differentiation, migration, invasion, and deposition of ECM. Among the potential targets, the receptor TGFβ-R1 is predicted to be targeted by the 3 sputum-dysregulated miRs (FIG. 5.B). The Database for Annotation, Visualization and Integrated Discovery (DAVID) (version 6.8) was used to visualize the direct targets belonging to TGF-β signaling pathway.

### Example 3. In Vitro Effects of miR-142-3p Containing Exosomes

Cell Culture and Treatments.

Human alveolar basal epithelial cell line (A549) and human lung fibroblast cell line (MRCS) were cultured in Minimum Essential Medium (MEM) (21090-055, Gibco) with 10% FBS, 2 mM L-glutamine (A2916801, ThermoFischer Scientific), 1% Non Essential Amino Acid (11140-035, Gibco) and 5 mM sodium pyruvate (11360-039, Gibco) for A549 only. THP-1 monocytes (ATCC) were cultured in RPMI1640 with L-glutamine and 10% FBS. To induce the differentiation of THP-1 monocytes into macrophage-like cells (THP-1 macrophages), THP-1 monocytes were incubated with phorbol 12-myristate 13-acetate (PMA) (P1585, Sigma) (100 ng/ml) for 2 days followed by 3 days of rest. A549 and MRCS were stimulated with 5 ng/ml of Transforming Growth Factor beta (TGF-β) for 4 h.

Transfection of Cells with microRNA Mimics.

200,000 cells were transfected in 6-well plates with 12.5 nM miR control or human miR-142-3p duplexes using DharmaFECT 4 transfection reagent (T-2004-03, Thermo Scientific) and analyzed after 48 h.

Design of the Synthetic microRNA.

As an example, miR-142-3p can be produced from a synthetic RNA duplex. The miR-142 duplex was designed according to the outline provided by the publication by Ørom and Lund (Ørom and Lund, (2007), Methods, 43:162-165). In summary, a 5′ phosphorylation of the miR-142-3p was introduced. The carrier strand (miR-142-reverse) was the complementary RNA sequence, which was also phosphorylated at the 5′ end and carried a UU 3′ overhang but contains mutations near the 3′ end to thermodynamically destabilize the strand and induce faster degradation. Oligonucleotides sense (has-miR-142-3p-S) and reverse (hsa-miR-142-3p-AS) were annealed and used as a duplex. The sequence and modifications introduced in the miR duplexes by Ørom and Lund are

hsa-miR-142-3p-S:
(SEQ ID NO: 1)
5′ UGUAGUGUUUCCUACUUUAUGGA 3′,
hsa-miR-142-3p-AS:
(SEQ ID NO: 3)
5′ CAUAAAGUAGGCAACAUCUACCUU 3′.
cel-miR-67-S:
(SEQ ID NO: 4)
5′ UCACAACCUCCUAGAAAGAGUAGA 3′
cel-miR-67-AS:
(SEQ ID NO: 5)
5′ UACUCUUUCUAGAAGGUGGUGCUU 3′

MiR-142-.3p Mimic Blunts TGF-β-Induced Fibrotic Response in Lung Epithelial Cells and Fibroblasts.

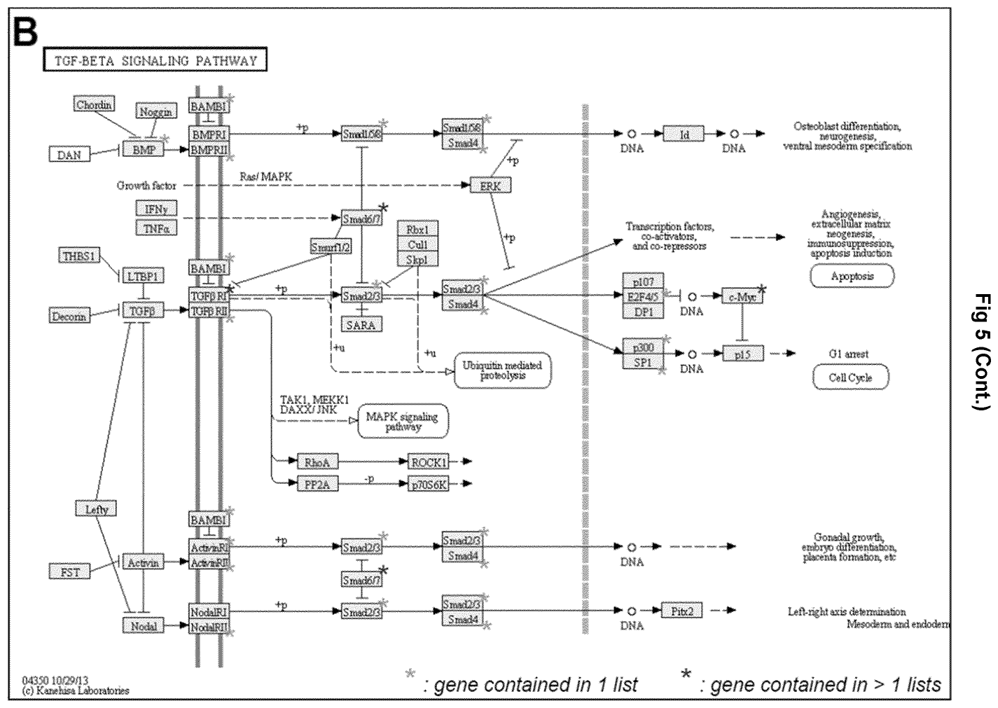

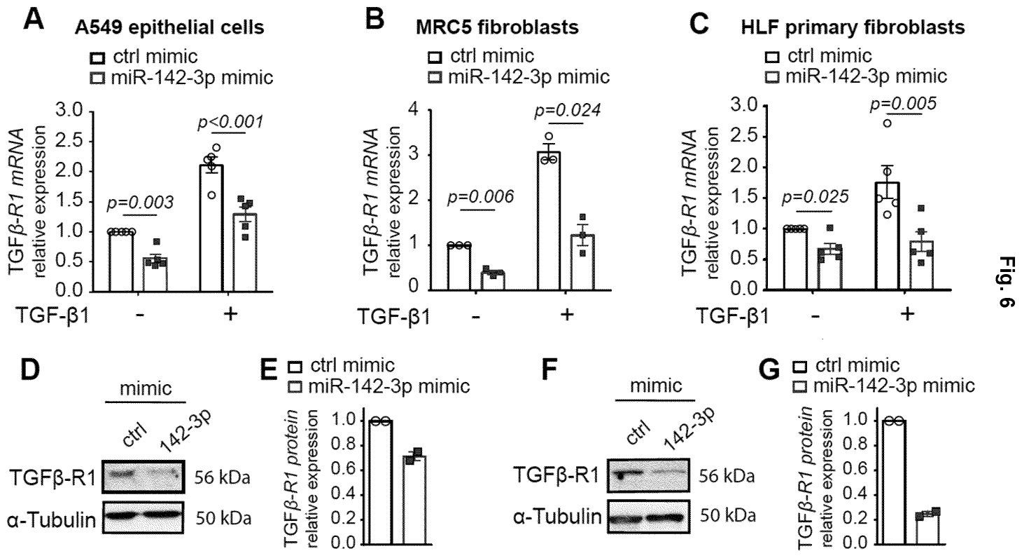

The biological role of miR-142-3p was investigated by transfecting alveolar epithelial cells (A549), lung fibroblasts (MRCS), and HLF primary fibroblasts with mimics. Interestingly, these results show that miR-142-3p was able to suppress the induction of pro-fibrotic genes (COL1A1, COL3A1 and TFG-β1) in A549, MRCS and HLF cells in presence of TGF-β (FIGS. 6.H, I, and J, respectively). Furthermore, the reduction of direct target TGFβ-R1 at mRNA and protein levels in A549 epithelial cells (FIG. 6.A, D, E), MRCS fibroblasts (FIG. 6.B, F, G), and HLF primary fibroblasts (FIG. 6.C) could be demonstrated. These data highlight the anti-fibrotic properties of miR-142-3p via the targeting of TGFβ-R1.

Macrophage-Derived Exosomes Transfer miR-142-.3p to Recipient Lungfibroblasts and Epithelial Cells.

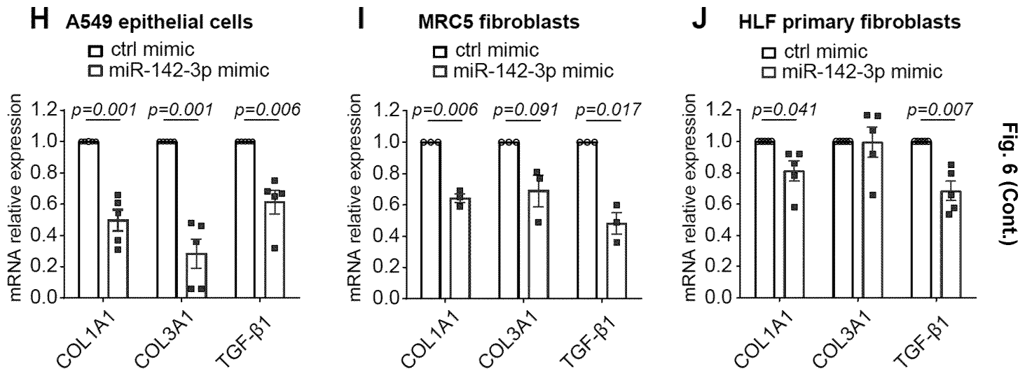

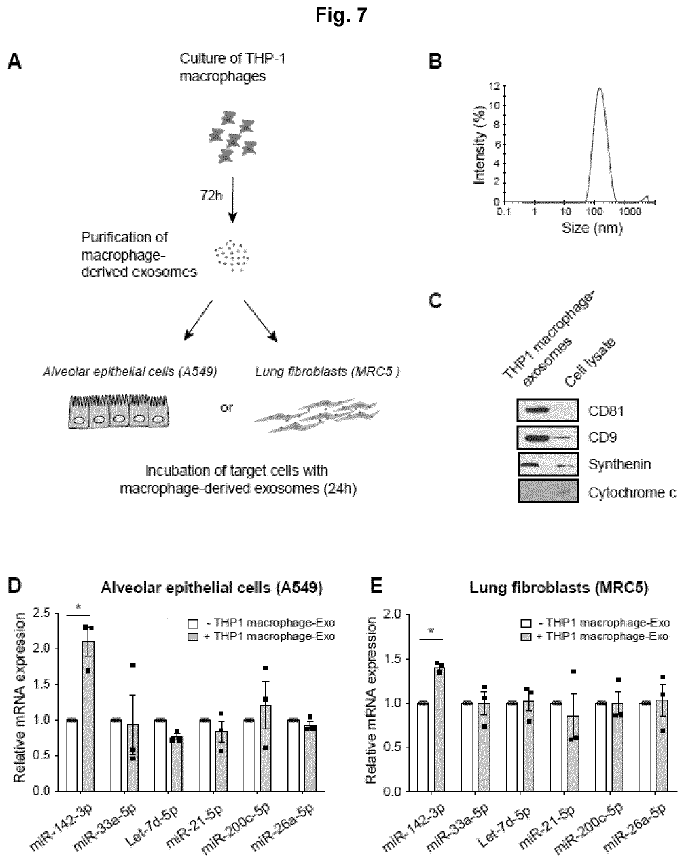

Because miR-142-3p is enriched in THP1 macrophages, it was tested whether this miR could be transferred to alveolar epithelial cells and fibroblasts via macrophage-derived exosomes. To this end, purified THP1 macrophages-exosomes were added to A549 and MRCS cells for 24 hours and the expression of IPF-related miRNAs was measured (FIG. 7.A-C). As expected, the level of miR-142-3p increases in A549 and MRCS cells in presence of macrophage-derived exosomes (2.1 fold, p=0.033; and 1.41 fold, p=0.034, respectively) (FIG. 7.D, E, respectively).

Macrophage-Derived Exosomes are Able to Suppress Profibrotic Epithelial Cell Activation.

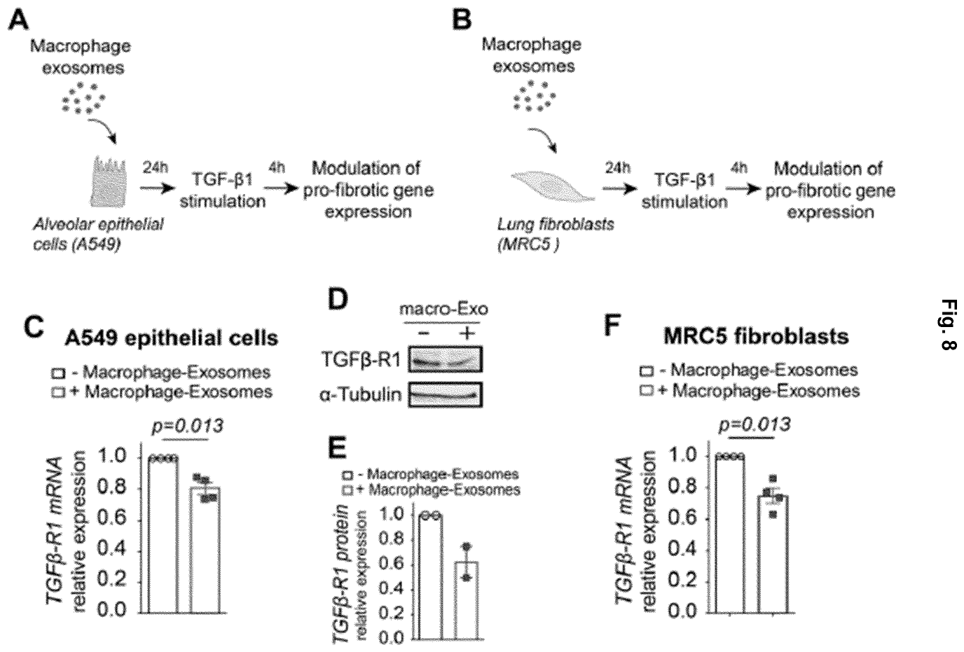

Because macrophage-exosomes are able to transfer anti-fibrotic miR-142-3p to target cells, the inventors anticipated that these extracellular vesicles present anti-fibrotic properties. To test this, purified THP1 macrophages-exosomes were added to A549 cells (FIG. 8.A) and to MRCS fibroblasts (FIG. 8.B) for 24 hours, followed by stimulation with TGF-τ3 for 4 h. Macrophage-exosomes suppressed the expression of the direct target TGFβ-R1 at mRNA and protein levels and the induction of pro-fibrotic genes (COL1A1, COL3A1 and TGF-β1) in A549 (FIG. 8.C, D, E, G) and MRC-5 cells (FIG. 8.F, H). Similar results are obtained with exosomes produced from the A549 alveolar epithelial cell line, the MRCS lung fibroblast cell line, and THP-1 monocytic cell line.

Macrophage-Derived Exosomes do not Impact Proinflammatory Genes.

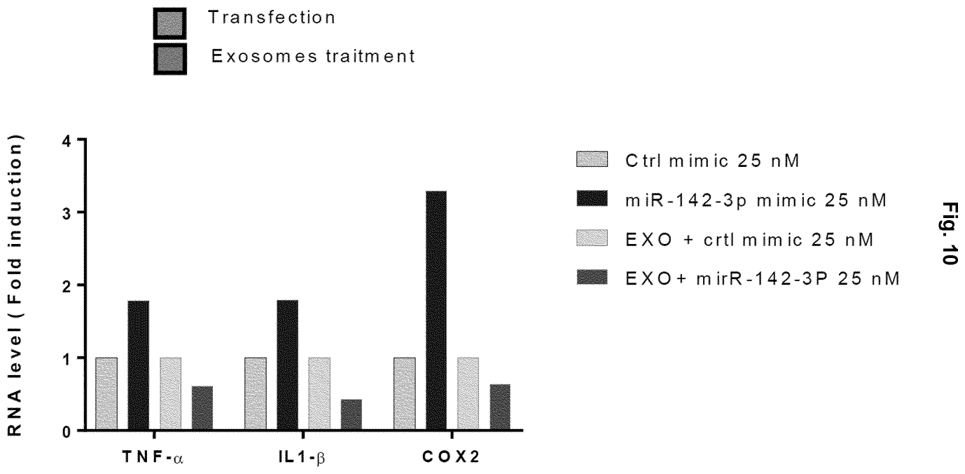

The inventors further investigated the impact of miR-142-3p exosomes on inflammation which is closely linked to fibrosis. Expression of TNFa, IL-10 and COX2 genes expression was analyzed by qRT-PCR. For quantification of mRNA expression by qRT-PCR, 500 ng RNA was transcribed into cDNA using the iScript cDNA Synthesis (Bio-Rad), and qRT-PCR was conducted in triplicate using Takyon MasterMix (Eurogentec). Data was normalized to Glyceraldehyde 3-phosphate dehydrogenase (GAPDH) using the Delta-Delta Ct method. All qRT-PCR primers are listed in Table 3 below. Surprisingly it could be observed that miR-142-3p packaged in exosomes does not upregulate proinflammatory genes TNFa, IL-1β and COX2, but rather inhibits them (FIG. 10). In contrast, when using miR-142-3p that was not packaged in exosomes a strong induction of pro-inflammatory genes could be observed.

TABLE 3
Primers used for qRT-PCR assays of
TNFa, IL-1β and COX2.

| | |
|---|---|
| hsa-TNFa-for | TCTCTCTAATCAGCCCTCTGG |
| hsa-TNFa-rev | ACTCGGGGTTCGAGAAGAT |
| HSA-IL-1β for | ACCTCCAGGGACAGGATATG |
| HSA-IL-1β rev | AACACGCAGGACAGGTACAG |
| HSA-cox 2 for | GAGTACCGCAAACGCTTTATGC |
| HSA-cox 2 rev | ATCTGGCCGAGGCTTTTCT |

Macrophage-Derived Loaded with miR-142-.3p Inhibitor Blocks the Anti-Fibrotic Properties of Exosomes.

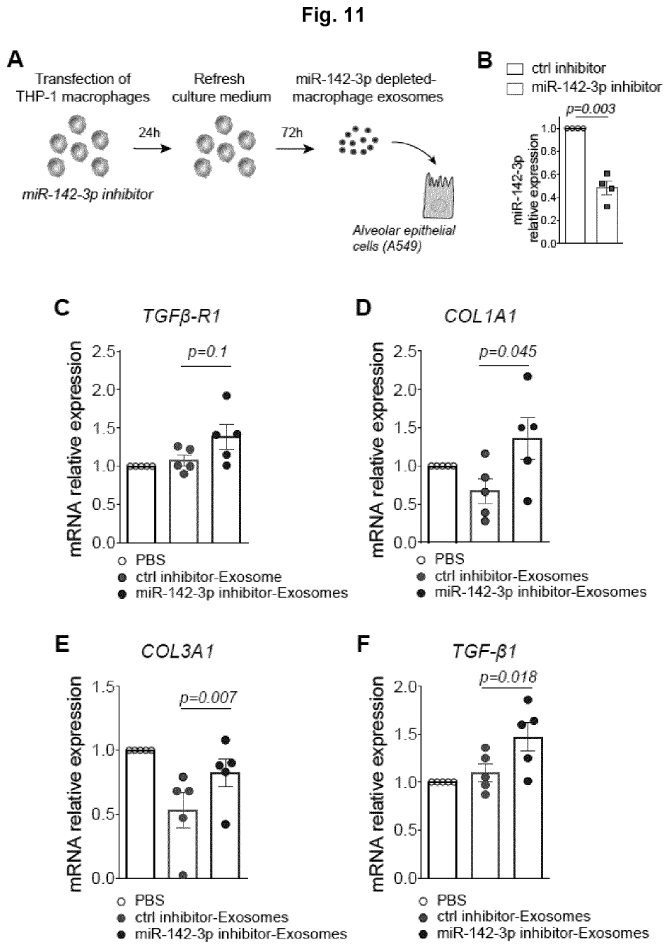

To further demonstrated the role of miR-142-3p in the antifibrotic properties of exosomes, the inventors loaded exosomes with miR-142-3p inhibitor (FIG. 11.A) and demonstrate that once the exosomes are added on A549 cells, an increase of TGFβ-R1 and profibrotic genes (COL1A1, COL3A1 and TGF-β1) is observed (FIG. 11.C-F). A depletion in miR-142-3p in A549 cells could be observed, in accordance with the experimental design (FIG. 11B).

### Example 4. In Vivo Assessment of miR-142-3p Containing Exosomes in a Mouse Model of Pulmonary Fibrosis

In Vivo Experiments.

All animal protocols were approved by the animal care committee at the University of Liege and the GIGA center (Liege, Belgium).

Cel-miR-67-Loaded Exosomes are Able to Deliver Cel-miR-67 In Vivo.

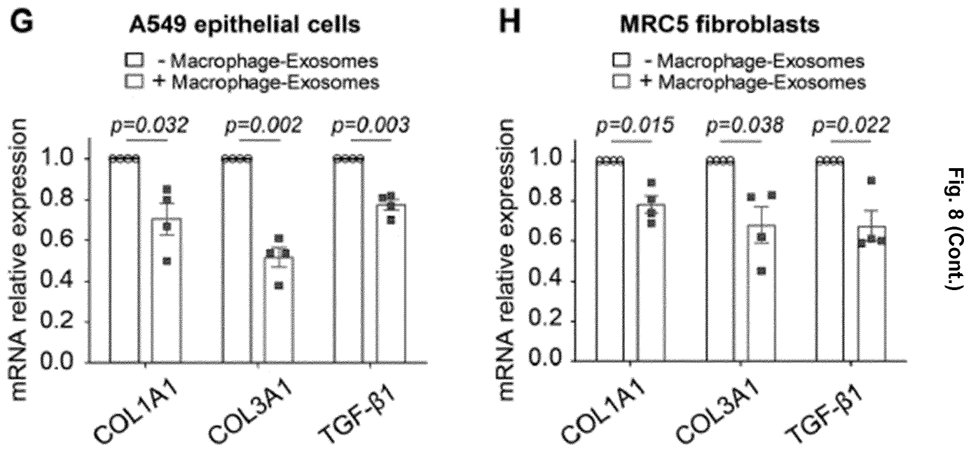

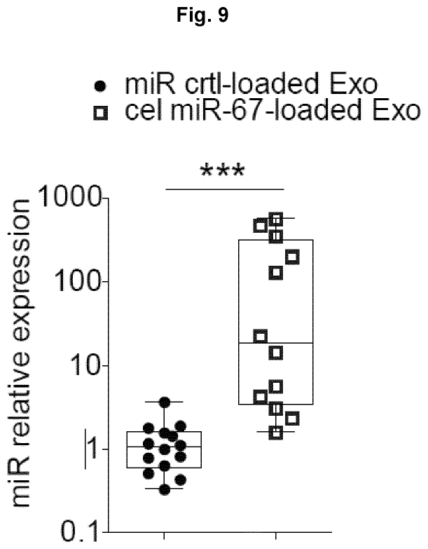

To investigate the effect of miR-142-3p in the progression of pulmonary fibrosis in vivo, they have used “miR-loaded exosomes” approach (previously design in the lab). These modified exosomes are able to deliver candidate miR in vivo (FIG. 9).

Endothelial exosomes loaded with an exogenous miR, cel-miR-67, were generated by means of electroporation and delivered in the tumor of mouse model of breast cancer. miR-67- or miR control-loaded exosomes were injected subcutaneously in the peritumoral region, and this operation was repeated every two days until day 21 (tumor<1 cm3). The presence of cel-miR-67 in tumor (146 fold, p<0.0001) demonstrated the capacity of electroporated exosomes to deliver miR mimics in vivo (FIG. 9).

Electroporating MS1-Derived Exosomes with Cel-miR-67.

Exosomes isolated from MS-1 mouse endothelial cell line were electroporated with cel-miR-67 or miR control mimic. For one mouse injection, 2 μg of miR-67- or control miR-loaded exosomes resuspended in 25 μl of PBS were used.

Increasing Mir-142-.3p Levels in Exosomes Increases the miR-142-.3p Anti-Fibrotic Activity.

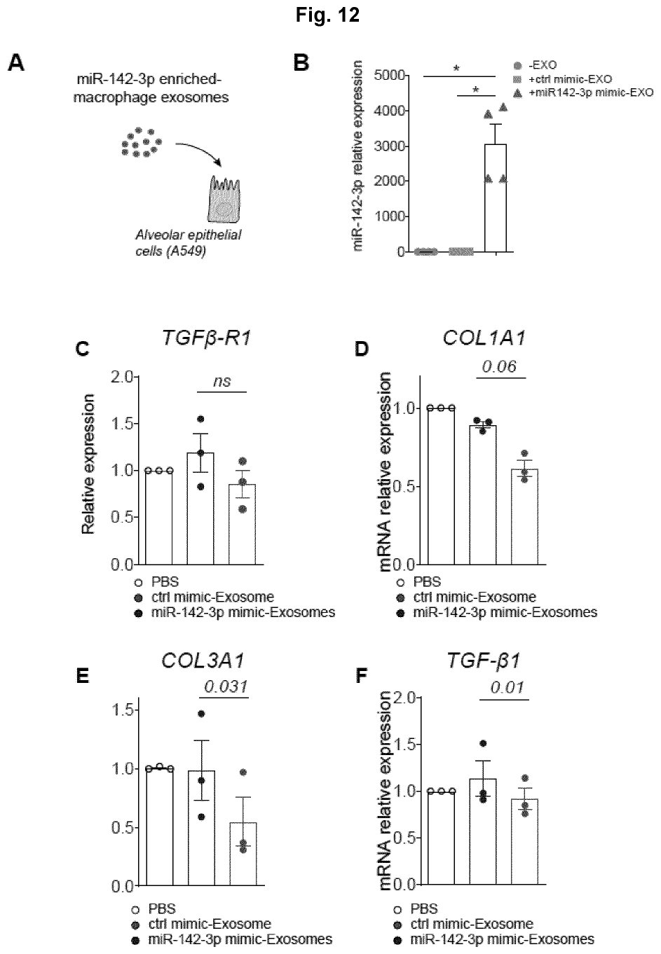

A549 cells were incubated with exosomes transfected with miR-142-3p (i.e. supplemented with additional miR-142-3p, FIG. 12.A) show a stronger anti-fibrotic effect when compared to their non-miR-142-3p enriched counterparts (FIG. 12.B). miR-142-3 mimic exosomes show a more pronounced repressive effect on TGFβ-R1 in A549 cells (FIG. 12.C) and pro-fibrotic genes (COL1A1, COL3A1, TGF-β1) (FIG. 12.D-F).

Evaluation of the Benefit of Using miR-142-3p Exosomes to Prevent Fibrosis in a Mouse Model of Pulmonary Fibrosis.

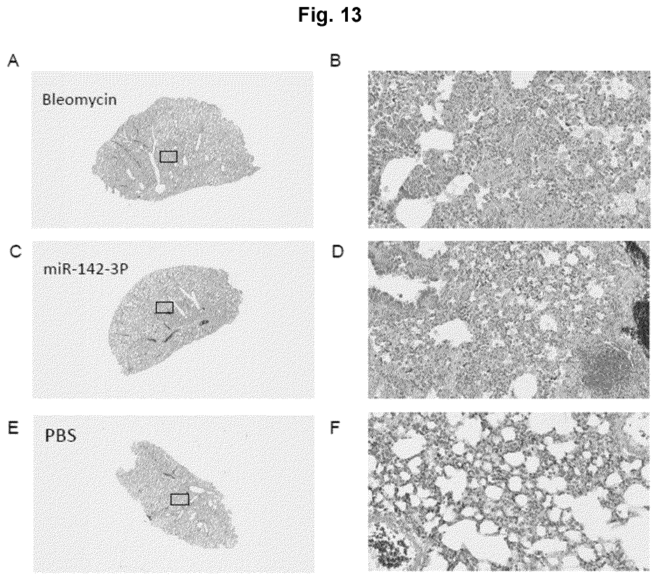

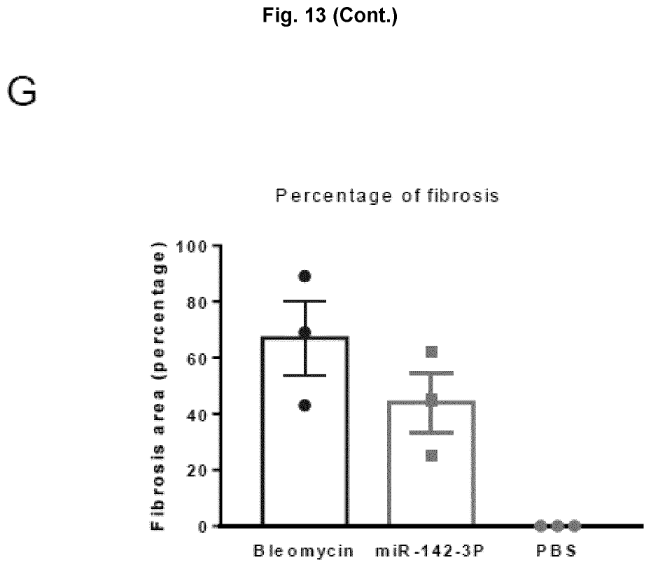

Among currently applied models of experimentally induced pulmonary fibrosis, the administration of bleomycin is frequently used (Moore et al., (2013), Am. J. Respir. Cell Mol. Biol., 49:167-179). Furthermore, bleomycin can be introduced by multiple methods, including intratracheal, intraperitoneal, subcutaneous, intravenous, and inhalation administration methods. To evaluate the anti-fibrotic effect of miR-142-3p packaged in exosomes in an in vivo experiment, mice were injected intratracheally with bleomycin on day 1 (FIG. 13.A-D) or PBS (FIG. 13.E-F). On day 7 and 14, panels (C-D) illustrate mice injected intratracheally with miR-142-3P mimic loaded exosomes. Magnification are 1.5× (panel A, C, E) and 20× (panel B, D, F). A profound and significant reduction in fibrosis could be observed in the miR-142-3p exosome condition when compared to the bleomycin treated condition that did not received the miR-142-3p exosome treatment (FIG. 13.G). Percentages in FIG. 13.G represent the area of fibrosis in relative to the total lung area. Remarkably, this experiment provides conclusive evidence that miR-142-3p acts independently of its anti-inflammatory effect. In this experiment miR-142-3p encapsulated in exosomes is added on day 7 after bleomycin injection, well after the inflammatory phase that is induced in this model by bleomycin, which only last around a week before onset of the fibrosis phase (as explained in detail in Moore et al, 2013 Am J Respir Cell Mol Bio, 49, 167).

Assessment of miR Delivery Via Extracellular Vesicles.

Female BALB/c mice (8 weeks old; 18-20 g) were injected subcutaneously with 200 μl of 4T1 breast cancer cell line (0.1*106) under anesthesia. At day 5 (+/−50 mm3 tumor size), miR-67- or miR control-loaded exosomes were injected subcutaneously in the peritumoral region, and this operation was repeated every two days. At ˜day 21 (<1 cm3), all mice were sacrificed under general anesthesia and 4T1 tumors were collected. Subsequently, miR-67 quantification was performed by qRT-PCR.

Bleomycin-Induced Mouse Model of Lung Fibrosis.

8 weeks-old C57Bl/6 mice received 0.075 units/kg of bleomycin intratracheally. 7 days later, mice received intratracheal injection of PBS, 5 μg of exosomes electroporated with miR-ctrl (cel-67 mimic) or with miR-142-3P. Mice received same treatment with exosomes 7 days later. On day 21 mice were sacrificed. The mouse lungs were fixed in formol OVN, then in ethanol 70% for an additional 24 h and embedded in paraffin before sectioning into 5 μm-thick slices. The sections were stained with hematoxylin and Masson's trichrome staining to assess the degree of fibrosis. An experienced pathologist reviewed the histological sections. Electroporating Raw 264-derived exosomes with cel-miR-67 and miR-142-3p. Exosomes isolated from RAW 264 macrophages were electroporated with cel-miR-67 (miR-control mimic) or miR142-3p mimic. Each mouse received an injection of 5 μg exosomes containing 2.5 μg of cel-miR-67- or miR-142-3P resuspended in 50 μl of PBS. Quantification was performed using Image J software.

Statistical Analysis.

Statistical analyses were performed using SPSS statistics for Windows software version 20 or GraphPad Prism version 5. A p-value of less than 0.05 was considered to be statistically significant. For the analysis of subject characteristics, the values are expressed as the mean±Standard Deviation (SD). Statistical significance was determined using an unpaired t test (continuous variables). For comparison of miRNA expression in exosomes from IPF patients vs HS, a Mann-Whitney U test was used. The correlation between miRNA levels and sputum cell counts (%) was expressed by Spearman's correlation.

1. A method of treating or preventing a fibro-inflammatory disease in a subject in need thereof, comprising administering to the subject an effective amount of a pharmaceutical formulation comprising extracellular vesicles produced by an in vitro cellular expression system, wherein the extracellular vesicles comprise an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1).

2. The method of claim 1, wherein said sequence has preferably at least 85% identity to the nucleic acid sequence of microRNA miR-142-3p (SEQ ID NO: 1), more preferably at least 90% identity to the nucleic acid sequence of microRNA miR-142-3p (SEQ ID NO: 1), more preferably at least 95% identity to the nucleic acid sequence of microRNA miR-142-3p (SEQ ID NO: 1).

3. The method of claim 1, wherein the extracellular vesicles are selected from the group of extracellular vesicles consisting of: microvesicles (MVs), exosomes, oncosomes, and apoptotic bodies.

4. The method of claim 1, wherein the extracellular vesicles are derived from monocytes, macrophages or fibroblasts.

5. The method of claim 1, wherein the fibro-inflammatory disease is selected from the group of fibro-inflammatory diseases consisting of: idiopathic pulmonary fibrosis, systemic fibrosis, non specific interstitial lung disease, connective tissue disease, sarcoidosis, fibrosing sarcoidosis, chronic hypersensitivity pneumonitis, asbestosis, dermatomyositis, polymyositis, antisynthetase syndrome, cryptogenic organizing pneumonia, pulmonary fibrosis with auto-immune features, combined pulmonary fibrosis and emphysema, rheumatoid arthritis, arthrosis, crohn disease as well as ulcero-hemorragic rectocolitis.

6. The method of claim 1, wherein the pharmaceutical formulation is administered to the subject by intratracheal, intrabronchial, subcutaneous, transdermic, intravenous, aerosolized, nasal, intramucosal, intra-articular, or sublingual administration.

7. The method of claim 1, wherein the expression levels of one or more pro-fibrotic genes selected from the group consisting of: COL1A1, COL3A3, and TGF-β1 is decreased by at least 15%, more preferably at least 25%, at least 50%, at least 75%, at least 90%, in a subject treated with the pharmaceutical formulation when compared to a subject not receiving any anti-fibrotic treatment.

8. The method of claim 1, wherein the expression level of one or more pro-inflammatory genes selected from the group consisting of: TNFa, IL-1β, and COX2 is decreased by at least 10%, preferably at least 20%, more preferably at least 30%, more preferably at least 50% in a subject treated with said pharmaceutical formulation when compared to a subject not treated with said pharmaceutical formulation.

9. The method of claim 1, wherein at least one additional pharmaceutical active ingredient is combined with the pharmaceutical formulation prior to administration.

10. The method of claim 9, wherein said at least one additional pharmaceutical active ingredient is present in the extracellular vesicles of the pharmaceutical formulation.

11. A process for obtaining a pharmaceutical formulation of claim 1 comprising following steps:
culturing an in vitro cellular expression system;
isolating extracellular vesicles from the cellular expression system; and
introducing an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1), into the extracellular vesicles.

12. A process for obtaining a pharmaceutical formulation of claim 1 comprising following steps:
culturing an in vitro cellular expression system wherein an oligonucleotide comprising a sequence having at least 75% identity to the nucleic acid sequence of microRNA miR-142-3p (SEQ ID NO: 1), or a functionally active fragment of the nucleic acid sequence of microRNA miR-142-3p (SEQ ID NO: 1) is present in the cellular expression system; and
isolating extracellular vesicles from the cellular expression system.

13. The process of claim 12, wherein the oligonucleotide is an exogenous oligonucleotide.

14. The process of claim 11, further comprising a step wherein an agent is provided to the cellular expression system that increases the activity of microRNA miR-142-3p and/or the level of microRNA miR-142-3p that is present in the extracellular vesicle, preferably wherein said agent increases the activity and/or the level of miR-142-3p with present in the vesicle with at least 10%, preferably at least 25%, more preferably at least 50%, more preferably at least 75%.

15. The process of claim 11 wherein the in vitro cellular expression system comprises, consists essentially of, or consists of monocytes, macrophages or fibroblasts cells.

16. The method of claim 3, wherein the extracellular vesicles are exosomes.

17. The method of claim 4, wherein the extracellular vesicles are derived from THP-1 monocytes, THP-1 macrophages or HLF fibroblasts.

18. The method of claim 5, wherein the fibro-inflammatory disease is idiopathic pulmonary fibrosis or systemic fibrosis.

19. The process of claim 15, wherein the in vitro cellular expression system comprises, consists essentially of, or consists of THP-1 monocytes, THP-1 macrophages or HLF primary fibroblasts.
